(19) United States
(12) Patent Application Publication
(10) Pub. No.: US 2002/0137781 A1
(43) Pub. Date: Sep. 26, 2002

(54) Preparation and use of enantiomer-pure 2,4-disubstituted oxazolines

(76) Inventors: Olivier Froelich, Kembs (FR); Jacques Bouvier, Neuchatel (CH)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/961,888

(22) Filed: Sep. 24, 2001

Related U.S. Application Data
(63) Continuation of application No. PCT/EP00/02641, filed on Mar. 24, 2000.

(30) Foreign Application Priority Data
Mar. 26, 1999 (CH) 583/99

(51) Int. Cl. A01N 43/76; C07D 263/08

(52) U.S. Cl. 514/374; 548/237

(57) ABSTRACT

Enantiomers of formula

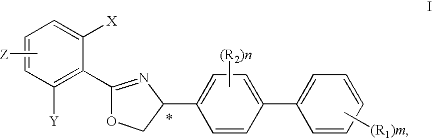

are described,

wherein X, Y, Z, R₁, R₂, m and n are defined as indicated in claim 1, each in free form or in salt form; a method of producing and the usage of these enantiomers; pesticides whose active ingredient is selected from these enantiomers; and a method of producing and the usage of these compositions.

This application is a continuation of PCT Patent Application No. PCT/EP00/02641, filed Mar. 24, 2000, which in its entirety is herein incorporated by reference.

The subject matter of the invention is enantiomers of formula

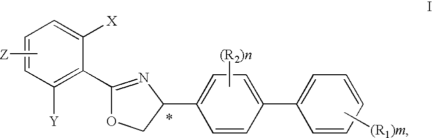

wherein

X and Y, independently of each other, are hydrogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, cyano-C₁-C₄-alkyl, cyano-C₁-C₄-haloalkyl, cyano-C₁-C₄-alkoxy, cyano-C₁-C₄-haloalkoxy, cyano-C₁-C₄-alkylthio, cyano-C₁-C₄-haloalkylthio, halogen, amino, cyano or nitro;

Z is hydrogen, halogen, C₁-C₄-alkyl, C₁-C₄-alkoxy or di(C₁-C₄-alkyl)amino;

R₁ is C₁-C₄-alkyl, C₁-C₄-haloalkyl, C₁-C₄-alkoxy, C₁-C₄-haloalkoxy, C₁-C₄-alkylthio, C₁-C₄-haloalkylthio, cyano-C₁-C₄-alkyl, cyano-C₁-C₄-haloalkyl, cyano-C₁-C₄-alkoxy, cyano-C₁-C₄-haloalkoxy, cyano-C₁-C₄-alkylthio, cyano-C₁-C₄-haloalkylthio, C₂-C₆-alkenyl, C₂-C₆-haloalkenyl, C₂-C₆-alkenyloxy, C₂-C₆-haloalkenyloxy, C₂-C₆-alkinyl, C₂-C₆-haloalkinyl, C₂-C₆-alkinyloxy, C₂-C₆-haloalkinyloxy, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, OC(O)R₃ or halogen;

R₂ is C₁-C₄-alkyl, C₁-C₄-haloalkyl, C₁-C₄-alkoxy, C₁-C₄-haloalkoxy, C₁-C₄-alkylthio, C₁-C₄-haloalkylthio or halogen;

R₃ is C₁-C₄-alkyl, C₁-C₄-alkoxy, C₂-C₆-alkenyl, C₂-C₆-alkenyloxy, C₂-C₆-alkinyl, C₂-C₆-alkinyloxy, C₃-C₈-cycloalkyl, C₃-C₈-cycloalkyloxy, C₃-C₈-halocycloalkyl-C₁-C₄-alkyl, C₃-C₈-halocycloalkyl-C₁-C₄-alkyloxy, N(R₄R₅) or unsubstituted or mono- to penta-substituted phenyl, whereby the substituents are selected from the group comprising C₁-C₄-alkyl, C₁-C₄-haloalkyl, C₁-C₄-alkoxy, C₁-C₄-haloalkoxy, C₁-C₄-alkylthio, C₁-C₄-haloalkylthio, halogen, cyano and nitro;

R₄ is hydrogen or C₁-C₄-alkyl;

R₅ is C₁-C₄-alkyl, C₁-C₄-haloalkyl, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, unsubstituted or mono to penta-substituted phenyl or unsubstituted or mono- to penta-substituted phenyl-C₁-C₄-alkyl, whereby independently of each other, the substituents are respectively selected from the group comprising C₁-C₄-alkyl; and

m and n, independently of each other, are 0, 1 or 2;

whereby when m or n is 2, R₁ or R₂ may be the same as one another or different, each respectively in free form or in salt form; a method or producing and the usage of these compounds and their salts; pesticides whose active ingredient is selected from these compounds; a method of producing and the usage of these compositions; and intermediates in free form or in salt form for the production of these compounds in free form or in salt form. The enantiomer mixtures of compound I are known from literature, for example from EP 0,432,661, EP 0,696,584 and DE 19,523,388, primarily for pest control in the field of crop protection. Despite their good efficacy, the properties of the known enantiomer mixtures when applied as pesticides are not always completely satisfactory against all pests, for which reason there is a need to provide compounds with improved pest-controlling properties, this problem being solved according to the invention by the preparation of the present substantially pure enantiomers of formula I.

Surprisingly, this need can be satisfied to a large extent by the usage of pure enantiomers of formula I, which are proposed according to the invention. It has been established that the respective enantiomer according to the invention, which is hereinafter called A, not only has improved, greater efficacy against pests than the enantiomer mixture, but also in addition, and just as unforeseeably, is in several cases better tolerated by the treated animals and plants than the enantiomer mixture, while the other enantiomer, hereinafter called B, shows no efficacy or very much lower efficacy against the pests. With increased efficacy of enantiomer A, there is a wider safety margin for the user, whereby the amount of active ingredient may be increased as required, in order to effectively control for example pests that are difficult to combat, without having to fear that the treated animal or the treated plant might be simultaneously harmed. The improved properties of enantiomer A makes it extremely interesting mixing partner for the combination with other active substances, e.g. to broaden the spectrum of activity. In the mixture both partners can be used in a substantially lower dose, and any disadvantageous interaction of the unnecessary, inactive enantiomer B with the partner in the mixture is excluded. Furthermore, from the perspective of a successful resistance management, it is advantageous to use the pure enantiomer A because the permanent presence of a sub-lethal dosage of the inactive enantiomer B could significantly speed up the development of resistance in the target pest.

In addition, the enantiomers of formula I are notable for their improved crystallization behavior and better formulation properties.

Usually, enantiomers A of formula I exhibit negative optical rotation in the polarized Na_{D} light (589 nm) of a sodium vapor lamp. However, enantiomers A with positive optical rotation should not be excluded. In any case, the significantly more active enantiomer is A.

Therefore, in accordance with the invention, enantiomers A of formula I are proposed as pesticides, especially to control insects and members of the order Acarina.

Preference is given to enantiomers, which are present in a purity of at least 95%.

The compounds of formula I may form salts, e.g. acid addition salts. These are formed for example with strong inorganic acids, such as mineral acids, e.g. sulphuric acid, a phosphoric acid or a hydrohalic acid, with strong organic carboxylic acids, such as C₁-C₄-alkane-carboxylic acids substituted where appropriate for example by halogen, e.g. acetic acid, such as optionally unsaturated dicarboxylic acids, e.g. oxalic, malonic, maleic, fumaric or phthalic acid, such as hydroxycarboxylic acids, e.g. ascorbic, lactic, malic, tartaric or citric acid, or benzoic acid, or with organic sulphonic acids, such as C₁-C₄alkanesulphonic or arylsulphonic acids substituted where appropriate for example by halogen, e.g. methanesulphonic or p-toluenesulphonic acid. The free form is preferred. Of the salts of the enantiomers of formula I, the agrochemically advantageous salts are preferred. Hereinbefore and hereinafter, the free enantiomers of formula I and their salts are understood where appropriate to include also by analogy the corresponding salts or free enantiomers of formula I.

Unless otherwise defined, the general terms used hereinabove and hereinbelow have the meanings given hereinbelow.

The halogen atoms considered as substituents of halogen-alkyl and halogen-alkoxy are fluorine, chlorine, bromine and iodine, with fluorine and chlorine being preferred.

If not defined to the contrary, carbon-containing groups and compounds contain preferably 1 to 4 inclusive, especially 1 or 2, carbon atoms.

Alkyl—as a group per se and as structural element of other groups and compounds such as alkoxy, halogen-alkyl or halogen-alkoxy—is, in each case with due consideration of the specific number of carbon atoms in the group or compound in question, either straight-chained or branched, and is methyl, ethyl, propyl, isopropyl, butyl, isobutyl, sec.-butyl or tert.-butyl or one of the respective isomers thereof. Preferred alkyl groups are C₁-C₂-alkyl groups, especially methyl groups.

Cycloalkyl—as a group per se and as structural element of other groups and compounds such as halocycloalkyl, cycloalkoxy and cycloalkylthio, —is, in each case with due consideration of the specific number of carbon atoms in the group or compound in question, cyclopropyl, cyclobutyl, cyclopentyl, cyclohexyl, cycloheptyl or cyclooctyl.

Alkenyl—as a group per se and as structural element of other groups and compounds, such as alkenyloxy—is, in each case with due consideration of the specific number of carbon atoms in the group or compound in question and of the conjugated or isolated double bonds—either straight-chained, e.g. allyl, 2-butenyl, 3-pentenyl, 1-hexenyl, 1-heptenyl, 1,3-hexadienyl or 1,3-octadienyl, or branched, e.g. isopropenyl, isobutenyl, isoprenyl, tert.-pentenyl, isohexenyl, isoheptenyl or isooctenyl.

Alkinyl—as a group per se and as structural element of other groups and compounds, such as alkinyloxy—is, in each case with due consideration of the specific number of carbon atoms in the group or compound in question and of the conjugated or isolated double bonds—either straight-chained, e.g. propargyl, 2-butinyl, 3-pentinyl, 1-hexinyl, 1-heptinyl, 3-hexen-1-inyl or 1,5-heptadien-3-inyl, or branched, e.g. 3-methylbut-1-inyl, 4-ethylpent-1-inyl, 4-methylhex-2-inyl or 2-methylhept-3-inyl.

Halogen-substituted groups, i.e. halogen-alkyl and halogen-alkoxy, may be partially halogenated or perhalogenated. Examples of halogen-alkyl—as a group per se and as a structural element of other groups and compounds, such as halogen-alkoxy—are methyl which is mono- to trisubstituted by fluorine, chlorine and/or bromine, such as CHF₂ or CF₃; ethyl which is mono- to penta-substituted by fluorine, chlorine and/or bromine, such as CH₂CH₂F, CH₂CF₃, CF₂CF₃, CF₂CCl₃, CF₂CHCl₂, CF₂CHF₂, CF₂CHCl₂, CF₂CHBr₂, CF₂CHClF, CF₂CHBrF or CClFCHClF; and propyl or isopropyl which is mono- to hepta-substituted by fluorine, chlorine and/or bromine, such as CH₂CHBrCH₂Br, CF₂CHFCF₃, CH₂CF₂CF₃ or CH(CF₃)₂.

The enantiomers in a purity of from about 95-100%, preferably 98-100%, are preferred within the scope of the invention. Said preferred enantiomers are

(1) Enantiomers of formula I, wherein

X and Y, independently of each other, are chlorine or fluorine, preferably fluorine, and Z is hydrogen;

(2) Enantiomers of formula I, wherein

R₁ is C₁-C₂-alkyl, C₁-C₂-haloalkyl, C₁-C₂-alkoxy, C₁-C₂-haloalkoxy, C₁-C₂-alkylthio, C₁-C₂-haloalkylthio, cyano-C₁-C₂-alkyl, cyano-C₁-C₂-haloalkyl, cyano-C₁-C₂-alkoxy, cyano-C₁-C₂-haloalkoxy, cyano-C₁-C₂-alkylthio or cyano-C₁-C₂-haloalkylthio; preferably methyl, halomethyl, halomethoxy, halomethylthio, cyanohalomethyl or cyanomethyl; most preferably methyl, trifluoromethyl, trifluoromethoxy, trifluoromethylthio, cyanomethyl or cyanodifluoromethyl

(3) Enantiomers of formula I, wherein

R₂ is hydrogen, C₁-C₂-alkyl, C₁-C₂-alkoxy or halogen; preferably hydrogen, methyl or halogen; most preferably hydrogen;

(4) Enantiomers of formula I, wherein

m is 1 or 2, preferably 1;

(5) Enantiomers of formula I, wherein

n is 0 or 1, preferably 0;

(6) Enantiomers of formula I, wherein

R₁ is C₁-C₂-alkyl, C₁-C₂-haloalkyl, C₁-C₂-alkoxy, C₁-C₂-haloalkoxy, C₁-C₂-alkylthio, C₁-C₂haloalkylthio, cyano-C₁-C₂-alkyl, cyano-C₁-C₂-haloalkyl, cyano-C₁-C₂-alkoxy, cyano-C₁-C₂-haloalkoxy, cyano-C₁-C₂-alkylthio or cyano-C₁-C₂-haloalkylthio;

R₂ is hydrogen, C₁-C₂-alkyl, C₁-C₂-alkoxy or halogen;

m is 1 or 2, and

n is 0 or 1;

(7) Enantiomers of formula I, wherein

R₁ is methyl, halomethyl, halomethoxy, halomethylthio, cyanohalomethyl or cyanomethyl;

R₂ is hydrogen, methyl or halogen;

m is 1, and

n is 0 or 1;

(8) Enantiomers of formula I, wherein

R₁ is methyl, trifluoromethyl, trifluoromethoxy, trifluoromethylthio, cyanomethyl or cyanodifluoro-methyl;

R₂ is hydrogen; and

m is 1.

The following enantiomers of formula I are especially preferred:

2-(2,6-difluorophenyl)-4-(4′-trifluoromethylbiphenyl-4-yl)-4,5-dihydrooxazole;

2-(2,6-difluorophenyl)-4-(4′-methylbiphenyl-4-yl)-4,5-dihydrooxazole;

2-(2,6-difluorophenyl)-4-(4′-trifluoromethoxybiphenyl-4-yl)-4,5-dihydrooxazole;

2-(2,6-difluorophenyl)-4-(4′-difluoromethoxybiphenyl-4-yl)-4,5-dihydrooxazole;

2-(2,6-difluorophenyl)-4-(4′-cyanodifluoromethoxybiphenyl-4-yl)-4,5-dihydrooxazole;

2-(2,6-difluorophenyl)-4-(4′-{1,1,2,2-tetrafluoroethoxy}-biphenyl -4-yl )-4,5-dihydrooxazole; and

2-(2-chloro-6-fluorophenyl)-4-(4′-trifluoromethoxybiphenyl-4-yl)-4,5-dihydrooxazole;

The enantiomers of formula I according to the invention may be obtained from the known enantiomer mixtures by using appropriate separation methods for enantiomers. Such methods are for example physical methods, such as fractional crystallization or chromatography, optionally on chiral stationary phases, as well as derivatisation with defined optically active adjuvants and separation of the enantiomer pairs thus obtained by the said separation processes. The pure optical antipodes are subsequently obtained from such isolated enantiomer derivatives by cleavage of the adjuvant. A further method of producing enantiomers from racemates is specific stereoselective synthesis from optionally optically active starting products.

It has now been found that the enantiomers of formula I are obtained by separation of the enantiomer mixtures using column chromatography on a chiral stationary phase with organic solvents or solvent mixtures, preferably alcohols, optionally mixed with hydrocarbons, most preferably ethanol or a mixture of isopropanol and hexane.

It has now surprisingly been found that the enantiomers of formula I can not only be used for plant protection, as in the case of the enantiomer mixtures, but are also eminently suitable for the prevention and cure of ecto- and endo-parasites on humans and preferably on livestock, domestic animals and pets.

It has unexpectedly emerged that the enantiomers A and B of formula I according to the invention not only slightly differ in their biocidal action, but have completely different biocidal acvitity. Enantiomer A is at least 100 to 1000 times more active than B, the activity of B being of no significant commercial value. The activity of B has no biological relevance, since when using B, too many parasites survive. In addition, usage of B should be avoided, since it can encourage the build-up of resistance. To sum up, this means that the activity of the enantiomer mixture stems exclusively from enantiomer A, and B makes no contribution. Moreover, the tolerance of A is many times greater than that of B. This makes it possible to achieve the same activity with a lower dosage of active ingredient as with the enantiomer mixtures, and the increased tolerance also enables higher doses to be used in order to be able to effectively control pests that are difficult to combat without harming the host plant or the host animal.

The animal pests include for example those:

of the order Lepidoptera for example

Acleris spp., Adoxophyes spp., Aegeria spp., Agrotis spp., Alabama argillaceae, Amylois spp., Anticarsia gemmatalis, Archips spp., Argyrotaenia spp., Autographa spp., Busseola fusca, Cadra cautella, Carposina nipponensis, Chilo spp., Choristoneura spp., Clysia ambiguella, Cnaphalocrocis spp., Cnephasia spp., Cochylis spp., Coleophora spp., Crocidolomia binotalis, Cryptophlebia leucotreta, Cydia spp., Diatraea spp., Diparopsis castanea, Earias spp., Ephestia spp., Eucosma spp., Eupoecilia ambiguella, Euproctis spp., Euxoa spp., Grapholita spp., Hedya nubiferana, Heliothis (Helicoverpa) spp., Hellula undalis, Hyphantria cunea, Keiferia lycopersicella, Leucoptera scitella, Lithocollethis spp., Lobesia botrana, Lymantria spp., Lyonetia spp., Malacosoma spp., Mamestra brassicae, Manduca sexta, Operophtera spp., Ostrinia nubilalis, Pammene spp., Pandemis spp., Panolis flammea, Pectinophora gossypiella, Phthorimaea operculella, Pieris rapae, Pieris spp., Plutella xylostella, Prays spp., Scirpophaga spp., Sesamia spp., Sparganothis spp., Spodoptera spp., Synanthedon spp., Thaumetopoea spp., Tortrix spp., Trichoplusia ni and Yponomeuta spp.;

of the order Coleoptera for example

Agriotes spp., Anthonomus spp., Atomaria linearis, Chaetocnema tibialis, Cosmopolites spp., Curculio spp., Dermestes spp., Diabrotica spp., Epilachna spp., Eremnus spp., Leptinotarsa decemlineata, Lissorhoptrus spp., Melolontha spp., Orycaephilus spp., Otiorhynchus spp., Phlyctinus spp., Popillia spp., Psylliodes spp., Rhizopertha spp., Scarabeidae, Sitophilus spp., Sitotroga spp., Tenebrio spp., Tribolium spp. and Trogoderma spp.;

of the order Orthoptera for example

Blatta spp., Blattella spp., Gryllotalpa spp., Leucophaea maderae, Locusta spp., Periplaneta spp. and Schistocerca spp.;

of the order Isoptera for example

Reticulitermes spp.;

of the order Psocoptera for example

Liposcelis spp.;

of the order Anoplura for example

Haematopinus spp., Linognathus spp., Pediculus spp., Pemphigus spp. and Phylloxera spp.;

of the order Mallophaga for example

Damalinia spp., Trichodectes spp. and Bovicola spp.

of the order Thysanoptera for example

Frankliniella spp., Hercinothrips spp., Taeniothrips spp., Thrips palmi, Thrips tabaci and Scirtothrips aurantii;

of the order Heteroptera for example

Cimex spp., Distantiella theobroma, Dysdercus spp., Euchistus spp. Eurygaster spp. Leptocorisa spp., Nezara spp., Piesma spp., Rhodnius spp., Sahlbergella singularis, Scotinophara spp. and Triatoma spp.;

of the order Homoptera for example

Aleurothrixus floccosus, Aleyrodes brassicae, Aonidiella spp., Aphididae, Aphis spp., Aspidiotus spp., Bemisia tabaci, Ceroplaster spp., Chrysomphalus aonidium, Chrysomphalus dictyospermi, Coccus hesperidum, Empoasca spp., Eriosoma larigerum, Erythroneura spp., Gascardia spp., Laodelphax spp., Lecanium corni, Lepidosaphes spp., Macrosiphus spp., Myzus spp., Nephotettix spp., Nilaparvata spp., Paratoria spp., Pemphigus spp., Planococcus spp., Pseudaulacaspis spp., Pseudococcus spp., Psylla spp., Pulvinaria aethiopica, Quadraspidiotus spp., Rhopalosiphum spp., Saissetia spp., Scaphoideus spp., Schizaphis spp., Sitobion spp., Trialeurodes vaporariorum, Trioza erytreae and Unaspis citri;

of the order Hymenoptera for example

Acromyrmex, Atta spp., Cephus spp., Diprion spp., Diprionidae, Gilpinia polytoma, Hoplocampa spp., Lasius spp., Monomorium pharaonis, Neodiprion spp., Solenopsis spp. and Vespa spp.;

of the order Diptera for example

Aedes spp., Antherigona soccata, Bibio hortulanus, Calliphora erythrocephala, Ceratitis spp., Chrysomyia spp., Culex spp., Cuterebra spp., Dacus spp., Dermatobia spp., Drosophila melanogaster, Fannia spp., Gastrophilus spp., Glossina spp., Haematobia spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis pomonella, Sciara spp., Stomoxys spp., Tabanus spp., Tannia spp. and Tipula spp.;

of the order Siphonaptera for example

Ceratophyllus spp., Xenopsylla cheopis, Ctenocephalides felis, Pulex spp. and Ctenocephalides canis;

of the order Thysanura for example

Lepisma saccharina and

of the order Acarina for example

Acarus siro, Aceria sheldoni, Aculus schlechtendali, Amblyomma spp., Argas spp., Boophilus spp., Brevipalpus spp., Bryobia praetiosa, Calipitrimerus spp., Chorioptes spp., Dermanyssus gallinae, Dermatophagoides spp., Dermacentor spp., Eotetranychus carpini, Eriophyes spp., Haemaphysalis spp., Hyalomma spp., Ixodes spp., Myobia spp., Myocoptes spp., Olygonychus pratensis, Omithodoros spp., Panonychus spp., Phyllocoptruta oleivora, Polyphagotarsonemus latus, Psorergates spp., Psoroptes spp., Rhipicephalus spp., Rhizoglyphus spp., Sarcoptes spp., Tarsonemus spp. and Tetranychus spp., Acarapis woodi, Cheylettiella parasitivorax, Cytodites nudus, Demodex spp., Knemidocoptes mutans, Otodectes cynotis, Varroa jacobsoni; from the class of the nematodes, for example, the families Filariidae and Setariidae and the genera Haemonchus, Trichostrongylus, Ostertagia, Nematodirus, Cooperia, Ascaris, Bunostumum, Oesophagostonum, Chabertia, Trichuris, especially Trichuris vulpis, Strongylus, Trichonema, Dictyocaulus, Capillaria, Strongyloides, Heterakis, Toxocara, especially Toxocara canis, Ascaridia, Oxyuris, Ancylostoma, especially Ancylostoma caninum, Uncinaria, Toxascaris and Parascaris; Dirofilaria, especially Dirofilaria immitis (heartworm).

The lifecycles of various parasites which can infest humans or animals are known to be very complex, which makes it extremely difficult to control the parasites. Ticks for example may feed exclusively from a single host or from several. They attach themselves to the host animal and feed off its blood. The females, when engorged, drop from the host animal and then lay a large number of eggs in a protected site of the surrounding environment. The developing larvae look for a new host animal, where they develop via the nymphal stage into adults, which in turn take a blood meal until engorged. Certain species feed on two and some on three hosts during their lifecycle.

Ticks of economic importance are above all those which belong to the genera Amblyomma, Boophilus, Hyalomma, Ixodes, Rhipicephalus and Dermacentor, especially the species Boophilus microplus and B. annulatus, and most especially B. microplus. They are responsible for the transmission of numerous diseases, which can affect humans and animals. The diseases which are mostly transmitted are bacterial, protozoan, rickettsial and viral. The pathogens of such diseases are transmitted especially by ticks, which feed on more than one host. These diseases can lead to the debilitation or even death of the host animals. In most cases they cause considerable economic damage, for example by diminishing the value of meat from livestock, damaging the usable skin, or reducing milk production.

Ticks of the above species are usually controlled by treating the infested animals with an acaricidally active composition depending on the type of infestation involved, i.e. by curative means. The occurrence of ticks, for example on pastureland, is heavily dependent, however, on seasonal weather conditions, and the ultimate infestation of the host animals itself depends also on their resistance to the ticks. This means that the preventive control of ticks is difficult and time-consuming, because it is difficult to estimate inter alia the degree of infestation by the parasites and the resistance of the animals to them. Furthermore, when attempting the preventive control of parasites, lengthy surveillance for possible infestation is necessary, which creates additional problems. Curative control of the parasites is not usually the primary aim because, at the time when the control begins to work, considerable damage has often already occurred.

Owing to the equally complex lifecycle of fleas, none of the known methods for controlling these parasites is entirely satisfactory, in particular because most of the known control methods focus on applying the active ingredient to the habitat in the flea's various development stages. This method is very complex and often unreliable, however, because of the different development stages which a flea goes through and which respond quite differently to different classes of substance.

The flea infestation of animals, in particular of dogs and cats, is accompanied by unpleasant effects not only for the animal being treated, but also for the animal keeper. These untoward effects can result in e.g. local irritation, troublesome pruritus, or even allergies, and often lead to intense scratching. Moreover, animals infested with fleas are constantly exposed to the risk of becoming infected with Dipylidium spp. (i.e. tapeworms, cestodes), which are transmitted by fleas.

Surprisingly, it has now been found that certain forms of application, for example topical application, but especially systemic administration of enantiomer A of formula I, where appropriate with the addition of one or more compounds from other substance classes, e.g. methoprene, hydroprene, dicyclanil and cythioate, or their salts, to potentiate the effect, can eliminate the said ectoparasites very rapidly and completely, thus intervening to block the complex development cycle of the parasites, and at the same time achieving an efficient control of the endoparasites. These compositions are even capable of exerting their excellent parasiticidal effect in full when given to the host animal systemically, i.e. orally, parenterally, subcutaneously, intramuscularly or intravenously. It is now possible, through selective periodic administration of these compounds, to break the depicted cycle of constant reinfestation of the host animals with the various parasites in a simple manner and to achieve a lasting eradication of the parasites. The parasites are either killed or prevented from reproducing, or the juvenile stages are prevented from developing and/or growing up and are no longer able to harm the host animal.

A further preferred object of the present invention is thus a method for the control of parasites in and on humans, domestic animals, livestock and pets, comprising a composition which contains at least one compound of formula I, or a veterinarily acceptable salt thereof, and is administered to the host animal orally, parenterally or by implant at a parasiticidally effective dose.

Essential to the invention is the fact that the composition of the invention is administered in such a way that the active ingredients which the composition comprises can be taken up in sufficient quantity with the blood of the host animal by endoparasites, ectoparasites and other parasites which can be regarded as vectors for the transmission of endoparasites, so that the eggs laid by the adult parasites and/or the larvae hatching therefrom are not able to develop.

This is achieved with the composition of the invention using different forms of application, e.g. through the oral administration of the composition comprising the active ingredients. In this case, formulated means e.g. in the form of a powder, a tablet, a granulate, a capsule, an emulsion, a foam, in micro-encapsulated form, etc., whereby as already mentioned, the preparation does not necessarily have to be given to the animal directly, but may also be conveniently mixed with its food. Of course, all compositions to be administered orally may contain further additives, in addition to conventional formulation excipients. These additives encourage willing consumption by the host animal, for example suitable odorous substances and flavorings. Because of its simple practicability, oral usage is one of the preferred subjects of the invention. A further type of application is parenteral usage, e.g. by subcutaneous or intravenous injection, topical application or as a long-term preparation (depot form) in the form of an implant or injection of microcapsules (so-called “microspheres”).

Oral application also includes e.g. administration of animal food, for example dog and cat food, which contains the active substances already mixed therein, e.g. as biscuits, as chews, as water-soluble capsules or tablets, in water-soluble form that can be dripped onto the food, or in other forms that can be mixed with the animal food. The implants also include all the devices, which can be inserted into the body of the animal in order to deliver the substance.

Percutaneous application forms include for example the subcutaneous, dermal, intramuscular and even intravenous administration of injectable forms. Apart from the usual injection syringes with needles, needleless systems and pour-on and spot-on formulations may also be expedient. By choosing a suitable formulation, it is possible to enhance the penetration power of the active ingredients through the living tissue of the animal, and to maintain its availability. This is of importance e.g. if one or more pooorly soluble active ingredients are used, the low solubility of which require a solubility-enhancing measure, since the body fluids of the animal are only able to dissolve small amounts of the substance at a time.

Furthermore, the active ingredients may also be present in a matrix formulation, which physically prevents their decomposition and maintains the availability of the active ingredients. This matrix formulation is injected into the body and remains there as a type of depot, from which the active ingredient is continuously released. Such matrix formulations are known to the person skilled in the art. These are generally waxy, semi-solid excipients, for example plant waxes and polyethylene glycols with a high molecular weight or copolymers of degradable polyesters.

Good availability of the active ingredients is also achieved by inserting an implant of the active substances into the animal. Such implants are widely used in veterinary medicine and often consist of silicone-containing rubber. Here, the active substances are dispersed in the solid rubber or are found in the inside of a hollow rubber element. Care must be taken that active substances are selected, which are soluble in the rubber implant, since they are first dissolved in the rubber and then continuously seep from the rubber material to the body fluids of the animal to be treated.

The rate of release of the active substances from the implant, and thus the time span during which the implant shows activity, is generally determined by the accuracy of measurement (amount of active ingredient in the implant) of the implant, the environment of the implant and the polymer formulation from which the implant is made.

The administration of the active ingredients by means of an implant represents a further preferred constituent of the present invention. This type of administration is extremely economical and effective, because a correctly dimensioned implant guarantees a constant concentration of the active substances in the tissue of the host animal. Nowadays, implants can be designed and implanted in a simple manner, so that they are in a position to deliver the active ingredients over some months.

The administration of veterinary medicine additives to animal food is best known in the field of animal health. Usually, first of all, a so-called premix is produced, in which the active substances are dispersed in a liquid or finely distributed in solid carriers. This premix can normally contain about 1 to 800 g of the substances per kg, depending on the desired end concentration in the food.

It is known moreover that active ingredients can be hydrolysed or their effects attenuated by the constituents of the feed. These active substances are routinely formulated in a protective matrix, e.g. in gelatin, before being added to the premix.

The compounds of formula I according to the invention may be used alone or in combination with other biocides. They may be combined with pesticides having the same sphere of activity e.g. to increase activity, or with substances having another sphere of activity e.g. to broaden the range of activity. It can also be sensible to add so-called repellents. If the range of activity is to be extended to endoparasites, e.g. wormers, the compounds of formula I are suitably combined with substances having endoparasitic properties. Of course, they can also be used in combination with antibacterial compositions. Since the compounds of formula I are adulticides, i.e. since they are effective in particular against the adult stage of the target parasites, the addition of pesticides which instead attack the juvenile stages of the parasites may be very advantageous. In this way, the greatest part of those parasites that produce great economic damage will be covered. Moreover, this action will contribute substantially to avoiding the formation of resistance. Many combinations may also lead to synergistic effects, i.e. the total amount of active ingredient can be reduced, which is desirable from an ecological point of view. Preferred groups of combination partners and especially preferred combination partners are named in the following, whereby combinations may contain one or more of these partners in addition to a compound of formula I.

Suitable partners in the mixture may be biocides, e.g. the insecticides and acaricides with a varying mechanism of activity, which are named in the following and have been known to the person skilled in the art for a long time, e.g. chitin synthesis inhibitors, growth regulators; active ingredients which act as juvenile hormones; active ingredients which act as adulticides; broad-band insecticides, broad-band acaricides and nematicides; and also the well known anthelminthics and insect- and/or acarid-deterring substances, said repellents or detachers.

Non-limitative examples of suitable insecticides and acaricides are:

| | |
|---|---|
| (I) | Aldicarb; |
| (II) | Azinphos-methyl; |
| (III) | Benfuracarb; |
| (IV) | Bifenthrin; |
| (V) | Buprofezin; |
| (VI) | Carbofuran; |
| (VII) | Dibutylaminothio; |
| (VIII) | Cartap; |
| (IX) | Chlorfluazuron; |
| (X) | Chlorpyrifos; |
| (XI) | Cyfluthrin; |
| (XII) | Lambda-Cy- |
| | halothrin; |
| (XIII) | Alpha- |
| | cypermethrin; |
| (XIV) | zeta- |
| | Cypermethrin; |
| (XV) | Deltamethrin; |
| (XVI) | Diflubenzuron; |
| (XVII) | Endosulfan; |
| (XVIII) | Ethiofencarb; |
| (XIX) | Fenitrothion; |
| (XX) | Fenobucarb; |
| (XXI) | Fenvalerate; |
| (XXII) | Formothion; |
| (XXIII) | Methiocarb; |
| (XXIV) | Heptenophos; |
| (XXV) | Imidacloprid; |
| (XXVI) | Isoprocarb; |
| (XXVII) | Methamidophos; |
| (XXVIII) | Methomyl; |
| (XXIX) | Mevinphos; |
| (XXX) | Parathion; |
| (XXXI) | Parathion-methyl; |
| (XXXII) | Phosalone; |
| (XXXIII) | Pirimicarb; |
| (XXXIV) | Propoxur; |
| (XXXV) | Teflubenzuron; |
| (XXXVI) | Terbufos; |
| (XXXVII) | Triazamate; |
| (XXXVIII) | Abamectin; |
| (XXXIX) | Fenobucarb; |
| (XL) | Tebufenozide; |
| (XLI) | Fipronil; |
| (XLII) | beta-Cyfluthrin; |
| (XLIII) | Silafluofen; |
| (XLIV) | Fenpyroximate; |
| (XLV) | Pyridaben; |
| (XLVI) | Fenazaquin; |
| (XLVII) | Pyriproxyfen; |
| (XLVIII) | Pyrimidifen; |
| (XLIX) | Nitenpyram; |
| (L) | NI-25, Acetamiprid; |
| (LI) | Avermectin B₁; |
| (LII) | an insect-active extract from a plant; |
| (LIII) | a preparation containing insect-active nematodes; |
| (LIV) | a preparation obtained from Bacillus subtilis; |
| (LV) | a preparation containing insect-active fungi; |
| (LVI) | a preparation containing insect-active viruses; |
| (LVII) | AC 303 630; |
| (LVIII) | Acephat; |
| (LIX) | Acrinathrin; |
| (LX) | Alanycarb; |
| (LXI) | Alphamethrin; |
| (LXII) | Amitraz; |
| (LXIII) | AZ 60541; |
| (LXIV) | Azinphos A; |
| (LXV) | Azinphos M; |
| (LXVI) | Azocyclotin; |
| (LXVII) | Bendiocarb; |
| (LXVIII) | Bensultap; |
| (LXIX) | Betacyfluthrin; |
| (LXX) | BPMC; |
| (LXXI) | Brofenprox; |
| (LXXII) | Bromophos A; |
| (LXXIII) | Bufencarb; |
| (LXXIV) | Butocarboxin; |
| (LXXV) | Butylpyridaben; |
| (LXXVI) | Cadusafos; |
| (LXXVII) | Carbaryl; |
| (LXXVIII) | Carbophenothion; |
| (LXXIX) | Chloethocarb; |
| (LXXX) | Chlorethoxyfos; |
| (LXXXI) | Chlormephos; |
| (LXXXII) | Cis-Res-methrin; |
| (LXXXIII) | Clocythrin; |
| (LXXXIV) | Clofentezin; |
| (LXXXV) | Cyanophos; |
| (LXXXVI) | Cycloprothrin; |
| (LXXXVII) | Cyhexatin; |
| (LXXXVIII) | Demeton M; |
| (LXXXIX) | Demeton S; |
| (XC) | Demeton-S-methyl; |
| (XCI) | Dichlofenthion; |
| (XCII) | Dicliphos; |
| (XCIII) | Diethion; |
| (XCIV) | Dimethoat; |
| (XCV) | Dimethylvinphos; |
| (XCVI) | Dioxathion; |
| (XCVII) | Edifenphos; |
| (XCVIII) | Emamectin; |
| (XCIX) | Esfenvalerat; |
| (C) | Ethion; |
| (CI) | Ethofenprox; |
| (CII) | Ethoprophos; |
| (CIII) | Etrimphos; |
| (CIV) | Fenamiphos; |
| (CV) | Fenbutatinoxid; |
| (CVI) | Fenothiocarb; |
| (CVII) | Fenpropathrin; |
| (CVIII) | Fenpyrad; |
| (CIX) | Fenthion; |
| (CX) | Fluzinam; |
| (CXI) | Flucycloxuron; |
| (CXII) | Flucythrinat; |
| (CXIII) | Flufenoxuron; |
| (CXIV) | Flufenprox; |
| (CXV) | Fonophos; |
| (CXVI) | Fosthiazat; |
| (CXVII) | Fubfenprox; |
| (CXVIII) | HCH; |
| (CXIX) | Hexaflumuron; |
| (CXX) | Hexythiazox; |
| (CXXI) | Iprobenfos; |
| (CXXII) | Isofenphos; |
| (CXXIII) | Isoxathion; |
| (CXXIV) | Ivermectin; |
| (CXXV) | Lambda- |
| | cyhalothrin; |
| (CXXVI) | Malathion; |
| (CXXVII) | Mecarbam; |
| (CXXVIII) | Mesulfenphos; |
| (CXXIX) | Metaldehyd; |
| (CXXX) | Metolcarb; |
| (CXXXI) | Milbemectin; |
| (CXXXII) | Moxidectin; |
| (CXXXIII) | Naled; |
| (CXXXIV) | NC 184; |
| (CXXXV) | Omethoat; |
| (CXXXVI) | Oxamyl; |
| (CXXXVII) | Oxydemethon M; |
| (CXXXVIII) | Oxydeprofos; |
| (CXXXIX) | Permethrin; |
| (CXL) | Phenthoat; |
| (CXLI) | Phorat; |
| (CXLII) | Phosmet; |
| (CXLIII) | Phoxim; |
| (CXLIV) | Pirimiphos M; |
| (CXLV) | Pirimiphos A; |
| (CXLVI) | Promecarb; |
| (CXLVII) | Propaphos; |
| (CXLVIII) | Prothiofos; |
| (CLIX) | Prothoat; |
| (CL) | Pyrachlophos; |
| (CLI) | Pyradaphenthion; |
| (CLII) | Pyresmethrin; |
| (CLIII) | Pyrethrum; |
| (CLIV) | RH 5992; |
| (CLV) | Salithion; |
| (CLVI) | Sebufos; |
| (CLVII) | Sulfotep; |
| (CLVIII) | Sulprofos; |
| (CXLIX) | Tebufenpyrad; |
| (CLX) | Tebupirimphos; |
| (CLXI) | Tefluthrin; |
| (CLXII) | Temephos; |
| (CLXIII) | Terbam; |
| (CLXIV) | Tetrachlor- |
| | vinphos; |
| (CLXV) | Thiafenox; |
| (CLXVI) | Thiodicarb; |
| (CLXVII) | Thiofanox; |
| (CLXVIII) | Thionazin; |
| (CLXIX) | Thuringiensin; |
| (CLXX) | Tralomethrin; |
| (CLXXI) | Triarthen; |
| (CLXXII) | Triazophos; |
| (CLXXIII) | Triazuron; |
| (CLXXIV) | Trichlorfon; |
| (CLXXV) | Triflumuron; |
| (CLXXVI) | Trimethacarb; |
| (CLXXVII) | Vamidothion; |
| (CLXXVIII) | Xylylcarb; |
| (CLXXIX) | YI 5301/5302; |
| (CLXXX) | Zetamethrin; |
| (CLXXXI) | DPX-MP062; |
| (CLXXXII) | RH-2485; |
| (CLXXXIII) | D 2341; |
| (CLXXXIV) | XMC (3,5-Xylyl- |
| | Methylcarbamat), |
| (CLXXXV) | Lufenuron |
| (CLXXXVI) | Fluazuron |
| (CLXXXVII) | Methoprene |
| (CLXXXVIII) | Hydroprene |
| (CLXXXIX) | Fenoxycarb |
| (CXC) | Chlorfenapyr or |
| (CXCI) | Spinosad |

Non-limitative examples of suitable anthelminthics are named in the following, a few representatives have insecticidal and acaricidal activity in addition to the anthelminthic activity, and are partly already in the above list.

(A1) Praziquantel=2-cyclohexylcarbonyl-4-oxo-1,2,3,6,7,11b-hexahydro-4H-pyrazino[2,1-α]isoquinoline

(A2) Closantel=3,5-diiodo-N-[5-chloro-2-methyl-4-(a-cyano-4-chlorbenzyl)phenyl]salicylamide

(A3) Triclabendazole=5-chloro-6-(2,3-dichlorphenoxy)-2-methylthio-1H-benzimidazole

(A4) Levamisol=L-(−)-2,3,5,6-tetrahydro-6-phenylimidazo[2,1b]thiazole

(A5) Mebendazole=(5-benzoyl-1H-benzimidazol-2-yl)carbamic acid methyl ester

(A6) Omphalotin=a macrocyclic fermentation product of the fungus Omphalotus olearius described in WO 97/20857

(A7) Abamectin=Avermectin B1

(A8) Ivermectin=22,23-dihydroavermectin B1

(A9) Moxidectin=5-O-demethyl-28-deoxy-25-(1,3-dimethyl-1-butenyl)-6,28-epoxy-23-(methoxyimino)-milbemycin B

(A10) Doramectin=25-cyclohexyl-5-O-demethyl-25-de(1-methylpropyl)-avermectin A1a

(A11) Milbemectin=mixture of milbemycin A3 and milbemycin A4

(A12) Milbemycinoxim=5-oxime of milbemectin

Non-limitative examples of suitable repellents and detachers are:

(R1) DEET (N,N-diethyl-m-toluamide)

(R2) KBR 3023 N-butyl-2-oxycarbonyl-(2-hydroxy)-piperidine

(R3) Cymiazole=N,-2,3-dihydro-3-methyl-1,3-thiazol-2-ylidene-2,4-xylidene

The said partners in the mixture are best known to specialists in this field. Most are described in various editions of the Pesticide Manual, The British Crop Protection Council, London, and others in the various editions of The Merck Index, Merck & Co., Inc., Rahway, N.J., USA or in patent literature. Therefore, the following listing is restricted to a few places where they may be found by way of example.

(I) 2-Methyl-2-(methylthio)propionaldehyde-O-methylcarbamoyloxime (Aldicarb), from The Pesticide Manual, 11^{th} Ed. (1997), The British Crop Protection Council, London, page 26;

(II) S-(3,4-dihydro-4-oxobenzo[d]-[1,2,3]-triazin-3-ylmethyl)O,O-dimethyl-phosphorodithioate (Azinphos-methyl), from The Pesticide Manual, 11^{th}Ed. (1997), The British Crop Protection Council, London, page 67;

(III) Ethyl-N-[2,3-dihydro-2,2-dimethylbenzofuran-7-yloxycarbonyl-(methyl)aminothio]-N-isopropyl-β-alaninate (Benfuracarb), from The Pesticide Manual, 11^{th}Ed. (1997), The British Crop Protection Council, London, page 96;

(IV) 2-Methylbiphenyl-3-ylmethyl-(Z)-(1RS)-cis-3-(2-chloro-3,3,3-trifluorprop-1-enyl)-2,2-dimethylcyclopropancarboxylate (Bifenthrin), from The Pesticide Manual, 11^{th}Ed. (1997), The British Crop Protection Council, London, page 118;

(V) 2-tert-Butylimino-3-isopropyl-5-phenyl-1,3,5-thiadiazian-4-one (Buprofezin), from The Pesticide Manual, 11^{th}Ed. (1997), The British Crop Protection Council, London, page 157;

(VI) 2,3-Dihydro-2,2-dimethylbenzofuran-7-yl-methylcarbamate (Carbofuran), from The Pesticide Manual, 11^{th}Ed. (1997), The British Crop Protection Council, London, page 186;

(VII) 2,3-Dihydro-2,2-dimethylbenzofuran-7-yl-(dibutylaminothio)methylcarbamate (Carbosulfan), from The Pesticide Manual, 11^{th}Ed. (1997), The British Crop Protection Council, London, page 188;

(VIII) S,S′-(2-Dimethylaminotrimethylene)-bis(thiocarbamate) (Cartap), from The Pesticide Manual, 11^{th}Ed. (1997), The British Crop Protection Council, London, page 193;

(IX) 1-[3,5-Dichloro-4-(3-chloro-5-trifluoromethyl-2-pyridyloxy)phenyl]-3-(2,6-difluorobenzoyl)-urea (Chlorfluazuron), from The Pesticide Manual, 11^{th}Ed. (1997), The British Crop Protection Council, London, page 213;

(X) O,O-Diethyl-O-3,5,6-trichloro-2-pyridyl-phosphorothioate (Chlorpyrifos), from The Pesticide Manual, 11^{th}Ed. (1997), The British Crop Protection Council, London, page 235;

(XI) (RS)-α-Cyano-4-fluoro-3-phenoxybenzyl-(1RS,3RS; 1RS,3RS)-3-(2,2-dichlorvinyl)-2,2-di-methylcyclopropancarboxylate (Cyfluthrin), from The Pesticide Manual, 11^{th}Ed. (1997), The British Crop Protection Council, London, page 293;

(XII) Mixture of (S)-α-cyano-3-phenoxybenzyl-(Z)-(1R,3R)-3-(2-chloro-3,3,3-trifluoropropenyl)-2,2-dimethylcyclopropancarboxylate and (R)-α-cyano-3-phenoxybenzyl-(Z)-(1R,3R)-3-(2-chloro-3,3,3-trifluorpropenyl)-2,2-dimethylcyclopropancarboxylate (Lambda-Cyhalothrin), from The Pesticide Manual, 11^{th}Ed. (1997), The British Crop Protection Council, London, page 300;

(XIII) Racemate consisting of (S)-α-cyano-3-phenoxybenzyl-(1R,3R)-3-(2,2-dichlorovinyl)-2,2-dimethylcyclopropanecarboxylate and (R)-α-cyano-3-phenoxybenzyl-(1S,3S)-3-(2,2-dichlorovinyl)-2,2-dimethylcyclopropanecarboxylate (Alpha-cypermethrin), from The Pesticide Manual, 11^{th}Ed. (1997), The British Crop Protection Council, London, page 308;

(XIV) a mixture of stereoisomers of (S)-α-cyano-3-phenoxybenzyl(1RS,3RS,1RS,3RS)-3-(2,2-dichlorovinyl)-2,2-dimethylcyclopropanecarboxylate (zeta-Cypermethrin), from The Pesticide Manual, 11^{th}Ed. (1997), The British Crop Protection Council, London, page 314;

(XV) (S)-α-cyano-3-phenoxybenzyl-(1R,3R)-3-(2,2-dibromovinyl)-2,2-dimethylcyclopropan-carboxylate (Deltamethrin), from The Pesticide Manual, 11^{th}Ed. (1997), The British Crop Protection Council, London, page 344;

(XVI) (4-Chlorophenyl)-3-(2,6-difluorbenzoyl)urea (Diflubenzuron), from The Pesticide Manual, 11^{th}Ed. (1997), The British Crop Protection Council, London, page 395;

(XVII) (1,4,5,6,7,7-Hexachloro-8,9,10-trinorborn-5-en-2,3-ylenebismethylene)-sulfite (Endosulfan), from The Pesticide Manual, 11^{th}Ed. (1997), The British Crop Protection Council, London, page 459;

(XVIII) α-Ethylthio-o-tolyl-methylcarbamate (Ethiofencarb), from The Pesticide Manual, 11^{th}Ed. (1997), The British Crop Protection Council, London, page 479;

(XIX) O,O-Dimethyl-O-4-nitro-m-tolyl-phosphorothioate (Fenitrothion), from The Pesticide Manual, 11^{th}Ed. (1997), The British Crop Protection Council, London, page 514;

(XX) 2-sec-Butylphenyl-methylcarbamate (Fenobucarb), from The Pesticide Manual, 11^{th}Ed. (1997), The British Crop Protection Council, London, page 516;

(XXI) (RS)-α-Cyano-3-phenoxybenzyl-(RS)-2-(4-chlorophenyl)-3-methylbutyrate (Fenvalerate), from The Pesticide Manual, 11^{th}Ed. (1997), The British Crop Protection Council, London, page 539;

(XXII) S-[formyl(methyl)carbamoylmethyl]-O,O-dimethyl-phosphorodithioate (Formothion), from The Pesticide Manual, 11^{th}Ed. (1997), The British Crop Protection Council, London, page 625;

(XXIII) 4-Methylthio-3,5-xylyl-methylcarbamate (Methiocarb), from The Pesticide Manual, 11^{th}Ed. (1997), The British Crop Protection Council, London, page 813;

(XXIV) 7-Chlorobicyclo[3.2.0]hepta-2,6-dien-6-yl-dimethylphosphate (Heptenophos), from The Pesticide Manual, 11^{th}Ed. (1997), The British Crop Protection Council, London, page 670;

(XXV) 1-(6-Chloro-3-pyridylmethyl)-N-nitroimidazolidin-2-ylidenamine (Imidacloprid), from The Pesticide Manual, 11^{th}Ed. (1997), The British Crop Protection Council, London, page 706;

(XXVI) 2-Isopropylphenyl-methylcarbamate (Isoprocarb), from The Pesticide Manual, 11^{th}Ed. (1997), The British Crop Protection Council, London, page 729;

(XXVII) O,S-Dimethyl-phosphoramidothioate (Methamidophos), from The Pesticide Manual, 11^{th}Ed. (1997), The British Crop Protection Council, London, page 808;

(XXVIII) S-methyl-N-(methylcarbamoyloxy)thioacetimidate (Methomyl), from The Pesticide Manual, 11^{th}Ed. (1997), The British Crop Protection Council, London, page 815;

(XXIX) Methyl-3-(dimethoxyphosphinoyloxy)but-2-enoate (Mevinphos), from The Pesticide Manual, 11^{th}Ed. (1997), The British Crop Protection Council, London, page 844;

(XXX) O,O-Diethyl-O-4-nitrophenyl-phosphorothioate (Parathion), from The Pesticide Manual, 11^{th}Ed. (1997), The British Crop Protection Council, London, page 926;

(XXXI) O,O-Dimethyl-O-4-nitrophenyl-phosphorothioate (Parathion-methyl), from The Pesticide Manual, 11^{th}Ed. (1997), The British Crop Protection Council, London, page 928;

(XXXII) S-6-chloro-2,3-dihydro-2-oxo-1,3-benzoxazol-3-ylmethyl-O,O-diethyl-phosphordithioate (Phosalone), from The Pesticide Manual, 11^{th}Ed. (1997), The British Crop Protection Council, London, page 963;

(XXXIII) 2-Dimethylamino-5,6-dimethylpyrimidin-4-yl-dimethylcarbamate (Pirimicarb), from The Pesticide Manual, 11^{th}Ed. (1997), The British Crop Protection Council, London, page 985;

(XXXIV) 2-Isopropoxyphenyl-methylcarbamate (Propoxur), from The Pesticide Manual, 11^{th}Ed. (1997), The British Crop Protection Council, London, page 1036;

(XXXV) 1-(3,5-dichloro-2,4-difluorophenyl)-3-(2,6-difluorobenzoyl)urea (Teflubenzuron), from The Pesticide Manual, 11^{th}Ed. (1997), The British Crop Protection Council, London, page 1158;

(XXXVI) S-tert-butylthiomethyl-O,O-dimethyl-phosphorodithioate (Terbufos), from The Pesticide Manual, 11^{th}Ed. (1997), The British Crop Protection Council, London, page 1165;

(XXXVII) Ethyl-(3-tert.-butyl-1-dimethylcarbamoyl-1H-1,2,4-triazol-5-yl-thio)-acetate, (Triazamate), from The Pesticide Manual, 11^{th}Ed. (1997), The British Crop Protection Council, London, page 1224;

(XXXVIII) Abamectin, from The Pesticide Manual, 11^{th}Ed. (1997), The British Crop Protection Council, London, page 3;

(XXXIX) 2-sec-butylphenyl-methylcarbamate (Fenobucarb), from The Pesticide Manual, 11^{th}Ed. (1997), The British Crop Protection Council, London, page 516;

(XL) N-tert.-butyl-N′-(4-ethylbenzoyl)-3,5-dimethylbenzohydrazide (Tebufenozide), from The Pesticide Manual, 11^{th}Ed. (1997), The British Crop Protection Council, London, page 1147;

(XLI) (±)-5-Amino-1-(2,6-dichloro-α,α,α-trifluoro-p-tolyl)-4-trifluoromethyl-sulfinylpyrazole-3-carbonitrile (Fipronil), from The Pesticide Manual, 11^{th}Ed. (1997), The British Crop Protection Council, London, page 545;

(XLII) (RS)-α-cyano-4-fluoro-3-phenoxybenzyl(1RS,3RS;1RS,3RS)-3-(2,2-dichlorovinyl)-2,2-dimethylcyclopropanecarboxylate (beta-Cyfluthrin), from The Pesticide Manual, 11^{th}Ed. (1997), The British Crop Protection Council, London, page 295;

(XLIII) (4-Ethoxyphenyl)-[3-(4-fluoro-3-phenoxyphenyl)propyl](dimethyl)silane (Silafluofen), from The Pesticide Manual, 11^{th}Ed. (1997), The British Crop Protection Council, London, page 1105;

(XLIV) tert.-butyl (E)-α-(1,3-dimethyl-5-phenoxypyrazol-4-yl-methylenamino-oxy)-p-toluate (Fenpyroximate), from The Pesticide Manual, ₁₁^{th}Ed. (1997), The British Crop Protection Council, London, page 530;

(XLV) 2-tert.-butyl-5-(4-tert.-butylbenzylthio)-4-chloropyridazin-3(2H)-one (Pyridaben), from The Pesticide Manual, 11^{th}Ed. (1997), The British Crop Protection Council, London, page 1161;

(XLVI) 4-[[4-(1,1-dimethylphenyl)phenyl]ethoxy]-quinazoline (Fenazaquin), from The Pesticide Manual, 11^{th}Ed. (1997), The British Crop Protection Council, London, page 507;

(XLVII) 4-Phenoxyphenyl-(RS)-2-(pyridyloxy)propyl-ether (Pyriproxyfen), from The Pesticide Manual, 11^{th}Ed. (1997), The British Crop Protection Council, London, page 1073;

(XLVIII) 5-Chloro-N-{2-[4-(2-ethoxyethyl)-2,3-dimethylphenoxy]ethyl}-6-ethylpyrimidine-4-amine (Pyrimidifen), from The Pesticide Manual, 11^{th}Ed. (1997), The British Crop Protection Council, London, page 1070;

(XLIX) (E)-N-(6-chloro-3-pyridylmethyl)-N-ethyl-N′-methyl-2-nitrovinylidenediamine (Nitenpyram), from The Pesticide Manual, 11^{th}Ed. (1997), The British Crop Protection Council, London, page 880;

(L) (E)-N¹-[(6-chloro-3-pyridyl)methyl]-N²-cyano-N¹-methylacetamidine (Nl-25, Acetamiprid), from The Pesticide Manual, 11^{th}Ed. (1997), The British Crop Protection Council, London, page 9;

(LI) Avermectin B₁, from The Pesticide Manual, 11^{th}Ed. (1997), The British Crop Protection Council, London, page 3;

(LII) an insect-active extract from a plant, especially (2R,6aS,12aS)-1,2,6,6a,12,12a-hexhydro-2-isopropenyl-8,9-dimethoxy-chromeno[3,4-b]furo[2,3-h]chromen-6-one (Rotenone), from The Pesticide Manual, 11^{th}Ed. (1997), The British Crop Protection Council, London, page 1097; and an extract from Azadirachta indica, especially Azadirachtin, from The Pesticide Manual, 11^{th}Ed. (1997), The British Crop Protection Council, London, page 59; and

(LII) a preparation which contains insect-active nematodes, preferably Heterorhabditis bacteriophora and Heterorhabditis megidis, from The Pesticide Manual, 11^{th}Ed. (1997), The British Crop Protection Council, London, page 671; Steinernema feltiae, from The Pesticide Manual, 11^{th}Ed. (1997), The British Crop Protection Council, London, page 1115, and Steinernema scapterisci, from The Pesticide Manual, 11^{th}Ed. (1997), The British Crop Protection Council, London, page 1116;

(LIV) a preparation obtainable from Bacillus subtilis, from The Pesticide Manual, 11^{th}Ed. (1997), The British Crop Protection Council, London, page 72; or from a strain of Bacillus thuringiensis with the exception of compounds isolated from GC91 or from NCTC11821; The Pesticide Manual, 11^{th}Ed. (1997), The British Crop Protection Council, London, page 73;

(LV) a preparation which contains insect-active fungi, preferably Verticillium lecanii, from The Pesticide Manual, 11^{th}Ed. (1997), The British Crop Protection Council, London, page 1266; Beauveria brogniartii, from The Pesticide Manual, 11^{th}Ed. (1997), The British Crop Protection Council, London, page 85 and Beauveria bassiana, from The Pesticide Manual, 11^{th}Ed. (1997), The British Crop Protection Council, London, page 83;

(LVI) a preparation which contains insect-active viruses, preferably Neodipridon Sertifer NPV, from The Pesticide Manual, 11^{th}Ed. (1997), The British Crop Protection Council, London, page 1342; Mamestra brassicae NPV, from The Pesticide Manual, 11^{th}Ed. (1997), The British Crop Protection Council, London, page 759; and Cydia pomonella granulosis virus, from The Pesticide Manual, 11^{th}Ed. (1997), The British Crop Protection Council, London, page 291;

(CLXXXI) 7-chloro-2,3,4a,5-tetrahydro-2-[methoxycarbonyl(4-trifluormethoxyphenyl) -carbamoyl]indol[1,2e]oxazoline-4a-carboxylate (DPX-MP062, Indoxycarb), from The Pesticide Manual, 11^{th}Ed. (1997), The British Crop Protection Council, London, page 453;

(CLXXXII) N′-tert.-butyl-N′-(3,5-dimethylbenzoyl)-3-methoxy-2-methylbenzohydrazide (RH-2485, Methoxyfenozide), from The Pesticide Manual, 11^{th}Ed. (1997), The British Crop Protection Council, London, page 1094; and

(CLXXXIII) (N′-[4-methoxy-biphenyl-3-yl]-hydrazinecarboxylic acid isopropyl ester (D 2341), from Brighton Crop Protection Conference, 1996, 487-493;

(R2) Book of Abstracts, 212th ACS National Meeting Orlando, Fla., Aug. 25-29 (1996), AGRO-020. Publisher: American Chemical Society, Washington, D.C. CONEN: 63BFAF.

As a consequence of the above details, a further essential aspect of the present invention relates to combination preparations for the control of parasites on warm-blooded animals, characterized in that they contain, in addition to a compound of formula I, at least one further active ingredient having the same or different sphere of activity and at least one physiologically acceptable carrier. The present invention is not restricted to two-fold combinations.

The compound of formula I is conveniently applied at a dosage of 0.01 to 800, preferably 0.1 to 200, especially 0.5 to 50 mg/kg body weight based on the humans or the host animal, oral administration being preferred.

A good dose of a compound of formula I which can be administered regularly to the host animal is especially 2.5-5 mg/kg bodyweight in the cat and 0.5-15 mg/kg per kg bodyweight in the dog. It is expedient to carry out the administration at regular intervals, e.g. every few days, weekly, or monthly.

The total dose can vary with the same active ingredient both between and within animal species, since the dose depends among other things on the weight and the constitution of the animal.

For the formulation of compositions that are to be administered to humans, domestic animals, livestock, and pets, the adjuvants known from veterinary practice for oral, parenteral and implant forms can be used. The following is a non-exhaustive list of some examples.

Suitable carriers are in particular fillers, such as sugars, e.g. lactose, saccharose, mannitol or sorbitol, cellulose preparations and/or calcium phosphates, e.g. tricalcium phosphate or calcium hydrogen phosphate, in a broader sense also binders, such as starch pastes using e.g. corn, wheat, rice or potato starch, gelatin, tragacanth, methyl cellulose and/or, if desired, disintegrants, such as the above-mentioned starches, in a broader sense also carboxymethyl starch, cross-linked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Tablet cores may be provided with suitable, where appropriate enteric, coatings, using inter alia concentrated sugar solutions which may comprise gum arabic, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes, flavours or pigments may be added to the tablets or tablet coatings, for example for identification purposes or to indicate different doses of active ingredient.

Further orally administrable pharmaceutical compositions include hard capsules consisting of gelatin, and also soft, sealed capsules consisting of gelatin and a plasticizer, such as glycerol or sorbitol. The hard capsules may contain the active ingredients in the form of granules, for example in admixture with fillers, such as lactose, binders, such as starches, and/or glidants, such as talc or magnesium stearate, and where appropriate stabilizers. In soft capsules, the active ingredients are preferably dissolved or suspended in suitable liquids, such as fatty oils, paraffin oil, or liquid polyethylene glycols, and stabilizers may likewise be added. Amongst other forms, capsules which can be both easily chewed and also swallowed whole are preferred.

The formulations suitable for parenteral administration are especially aqueous solutions of the active ingredients in water-soluble form, e.g. water-soluble salts, in the broader sense also suspensions of the active ingredients, such as appropriate oily injectable suspensions using suitable lipophilic solvents or vehicles, such as oils, e.g. sesame oil, or synthetic fatty acid esters, e.g. ethyl oleate, or triglycerides, or aqueous injectable suspensions containing viscosity-increasing agents, e.g. sodium carboxymethyl cellulose, sorbitol and/or dextran, and where appropriate stabilizers.

The compositions of the present invention may be prepared in a manner known per se, for example by means of conventional mixing, granulating, coating, dissolving or lyophilizing processes. Pharmaceutical compositions for oral administration can be obtained, for example, by combining the active ingredients with solid carriers, granulating a resulting mixture where appropriate, and processing the mixture or granules, if desired or necessary, to form tablets or tablet cores following the addition of suitable excipients.

The use of compounds of formula I according to the invention for the protection of plants against parasitic pests forms a particular focus of the present invention.

Pests of said type which occur on plants, especially on crops and ornamentals in agriculture, horticulture and forestry, or on parts of such plants, such as fruits, blooms, leaves, stems, tubers or roots, can be controlled, i.e. kept in check or eradicated, using the active ingredients of the invention, this protection remaining for parts of some plants whose growth does not occur until later.

Target crops include especially cereals, such as wheat, barley, rye, oats, rice, corn or sorghum; beet, such as sugar beet or fodder beet; fruit, e.g. pomes, drupes and soft fruit, such as apples, pears, plums, peaches, almonds, cherries or berries, e.g. strawberries, raspberries or blackberries; leguminous plants, such as beans, lentils, peas or soybean; oleaginous fruits, such as rape, mustard, poppy, olives, sunflowers, coconut, castor oil plants, cocoa beans or groundnuts; cucumber plants, such as squashes, cucumbers or melons; fibrous plants, such as cotton, flax, hemp or jute; citrus fruits, such as oranges, lemons, grapefruit or mandarins; vegetables, such as spinach, lettuce, asparagus, cabbages, carrots, onions, tomatoes, potatoes or paprika; lauraceae, such as avocado, cinnamon or camphor; and tobacco, nuts, coffee, aubergines, sugar cane, tea, pepper, vines, hops, banana plants, natural rubber plants and ornamentals.

The active ingredients of the invention are especially suitable for controlling Nila parvata lugens, Heliothis virescens, Spodoptera littoralis, Diabrotica balteata, Panonychus ulmi and Tetranychus urticae in vegetable, fruit, and rice crops.

Other indication areas for the active ingredients of the invention are the protection of stored products and stores and of material and, in the hygiene sector, especially the protection of domestic animals and livestock against pests of said type.

The invention therefore relates also to pesticides, such as emulsifiable concentrates, suspension concentrates, ready-to-spray or ready-to-dilute solutions, coatable pastes, dilute emulsions, spray powders, soluble powders, dispersible powders, wettable powders, dusts, granulates or encapsulations in polymeric substances, chosen in accordance with the intended objectives and prevailing circumstances, comprising at least one active ingredient of the invention.

The active ingredient is used in these compositions in pure form and a solid active ingredient e.g. in a specific particle size, or preferably together with—at least—one of the adjuvants conventionally employed in the art of formulation, such as extenders, e.g. solvents or solid carriers, or surface-active compounds (surfactants). For parasite control in humans, domestic animals, livestock, and pets of course only physiologically acceptable adjuvants are used.

In crop protection, suitable solvents include for example: aromatic hydrocarbons, partially hydrogenated where necessary, preferably fractions of alkylbenzenes having 8 to 12 carbon atoms, such as xylene mixtures, alkylated naphthalene or tetrahydronaphthalene, aliphatic or cyclo-aliphatic hydrocarbons, such as paraffins or cyclohexane, alcohols, such as ethanol, propanol or butanol, glycols and their ethers and esters, such as propylene glycol, dipropylene glycol ether, ethyl glycol or ethylene glycol monomethyl or ethyl ether, ketones, such as cyclohexanone, isophorone or diacetanol alcohol, strongly polar solvents, such as N-methylpyrrolid-2-one, dimethyl sulphoxide or N,N dimethylformamide, water, vegetable oils epoxidized where appropriate, such as rape, castor, coconut, or soybean oil epoxidized where appropriate, and silicone oils.

The solid carriers used e.g. for dusts and dispersible powders, are normally natural mineral fillers such as calcite, talcum, kaolin, montmorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite, and suitable non-sorbent carriers are materials such as calcite or sand. In addition, a great number of pregranulated materials of inorganic or organic nature can be used, e.g. especially dolomite or pulverized plant residues.

Depending on the nature of the active ingredient to be used in the formulation, suitable surface-active compounds are non-ionic, cationic and/or anionic surfactants having good emulsifying, dispersing and wetting properties. The surfactants specified below are to be regarded only as examples; the relevant literature describes many other surfactants that are commonly used in formulation technology and are suitable according to the invention.

Non-ionic surfactants are preferably polyglycol ether derivatives of aliphatic or cycloaliphatic alcohols, or saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols. Further suitable non-ionic surfactants are the water-soluble adducts of polyethylene oxide with polypropylene glycol, ethylenediamine propylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit. Suitable non-ionic surfactants are nonylphenolpolyethoxyethanols, castor oil polyglycol ethers, polypropylene/polyethylene oxide adducts, tributylphenoxypolyethoxyethanol, polyethylene glycol and octylphenoxypolyethoxyethanol. Also suitable are fatty acid esters of polyoxyethylene sorbitan, such as polyoxyethylene sorbitan trioleate.

Cationic surfactants are preferably quaternary ammonium salts which have as substituent at least one C₈-C₂₂ alkyl radical and, as further substituents, lower—where appropriate—halogenated alkyl, benzyl or lower hydroxyalkyl radicals. The salts are preferably in the form of halides, methylsulphates or ethylsulphates. Examples are stearyltrimethylammonium chloride and benzyl-di(2-chloroethyl)ethylammonium bromide.

Suitable anionic surfactants can be both water-soluble soaps and water-soluble synthetic surfactant compounds. Suitable soaps are the alkali metal salts, alkaline earth metal salts or unsubstituted or substituted ammonium salts of higher fatty acids (C₁₀-C₂₂), for example the sodium or potassium salts of oleic or stearic acid, or of natural fatty acid mixtures which can be obtained for example from coconut oil or tallow oil; the fatty acid methyltaurin salts may also be used. More frequently, however, synthetic surfactants are used, especially fatty sulphonates, fatty sulphates, sulphonated benzimidazole derivatives or alkylarylsulphonates. The fatty sulphonates or sulphates are usually in the form of alkali metal salts, alkaline earth metal salts or unsubstituted or substituted ammoniums salts and have an alkyl radical with 8 to 22 carbon atoms, which also includes the alkyl moiety of acyl radicals, for example, the sodium or calcium salt of lignonsulphonic acid, of dodecylsulphate or of a mixture of fatty alcohol sulphates obtained from natural fatty acids. These compounds also comprise the salts of sulphuric acid esters and sulphonic acids of fatty alcohol/ethylene oxide adducts. The sulphonated benzimidazole derivatives preferably contain 2 sulphonic acid groups and one fatty acid radical containing 8 to 22 carbon atoms. Examples of alkylarylsulphonates are the sodium, calcium or triethanolamine salts of dodecylbenzenesulphonic acid, dibutylnapthalenesulphonic acid, or of a naphthalenesulphonic acid/formaldehyde condensation product. Also suitable are corresponding phosphates, e.g. salts of the phosphoric acid ester of an adduct of p-nonylphenol with 4 to 14 moles of ethylene oxide or phospholipids.

By the term active ingredient is understood, hereinafter, enantiomer A, preferably an enantiomer A from the following substance table.

The compositions for use in crop protection and in humans, domestic animals, livestock, and pets usually contain 0.1 to 99%, especially 0.1 to 95%, of active ingredient and 1 to 99.9%, especially 5 to 99.9%,—at least—of a solid or liquid adjuvant, usually 0 to 25%, especially 0.1 to 20%, of the composition comprising surfactants (% in each case means percent by weight). Whereas concentrated compositions tend to be preferred for commercial goods, the end consumer as a rule uses dilute compositions which have substantially lower concentrations of active ingredient.

The composition of preferred crop protection agents is especially as follows (%=percent by weight):

Emulsifiable concentrates:

| | |
|---|---|
| active ingredient | 1 to 90%, preferably 5 to 20% |
| surfactant: | 1 to 30%, preferably 10 to 20% |
| solvent: | 5 to 98%, preferably 70 to 85% |
| Dusts: | |
| active ingredient: | 0,1 to 10%, preferably 0,1 to 1% |
| solid carrier: | 99.9 to 90%, preferably 99.9 to 99% |
| Suspension concentrates: | |
| active ingredient: | 5 to 75%, preferably 10 to 50% |
| water: | 94 to 24%, preferably 88 to 30% |
| surfactant: | 1 to 40%, preferably 2 to 30% |
| Wettable powders: | |
| active ingredient: | 0.5 to 90%, preferably 1 to 80% |
| surfactant: | 0.5 to 20%, preferably 1 to 15% |
| solid carrier: | 5 to 99%, preferably 15 to 98% |
| Granulates: | |
| active ingredient: | 0.5 to 30%, preferably 3 to 15% |
| solid carrier: | 99.5 to 70%, preferably 97 to 85% |

The activity of the crop protection agents of the invention can be substantially broadened and adapted to prevailing circumstances by adding other insecticidal substances. Additional active ingredients are, for example, substances from the following classes: organic phosphorus compounds, nitrophenols and their derivatives, formamidines, acylureas, carbamates, pyrethroids, nitroenamines and their derivatives, pyrroles, thioureas and their derivatives, chlorinated hydrocarbons and Bacillus thuringiensis preparations. The compositions of the invention can also contain further solid or liquid adjuvants, such as stabilizers, e.g. vegetable oils, epoxidized where appropriate (e.g. epoxidized coconut oil, rapeseed oil or soya oil), antifoaming agents, e.g. silicone oil, preservatives, viscosity modulators, binders and/or tackifiers, as well as fertilizers or other active ingredients to achieve specific effects, e.g. acaricides, bactericides, fungicides, nematocides, molluscicides or selective herbicides.

The crop protection agents of the invention are prepared in a known manner, in the absence of adjuvants e.g. by grinding, sieving, and/or compressing a solid active ingredient or active ingredient mixture, e.g. to a specific particle size, and in the presence of at least one adjuvant, e.g. by intimate mixing and/or grinding of the active ingredient or active ingredient mixture with the adjuvant(s). These methods for preparing compositions of the invention and the use of compounds of the formula I for preparing these compositions likewise form an object of the invention.

The methods of applying the crop protection agents, i.e. the methods for controlling pests of said type, such as spraying, atomizing, dusting, coating, dressing, scattering or pouring (chosen in accordance with the intended objectives and prevailing circumstances), and the use of the compositions for controlling pests of said type are further objects of the invention. Typical concentrations of active ingredient are between 0.1 and 1000 ppm, preferably between 0.1 and 500 ppm. The rates of application are generally 1 to 2000 g of active ingredient per hectare, especially 10 to 1000 g/ha, and preferably 20 to 600 g/ha.

A preferred method of application for crop protection is to apply the active ingredient to the foliage of the plants (leaf application), the number of applications and the rate of application depending on the intensity of infestation by the pest in question. However, the active ingredients can also penetrate the plant through the roots via the soil (systemic action) by impregnating the locus of the plant with a liquid composition, or by applying the compounds in solid form to the soil, e.g. in granular form (soil application). With paddy rice cultures, granules may be metered into the flooded paddy field.

The crop protection agents of the invention are also suitable for protecting vegetative propagation material, e.g. seeds, such as fruits, tubers or grains, or plant seedlings, from animal pests. The propagation material can be treated with the composition before the start of cultivation, seeds for example being dressed before they are sown. The active ingredients of the invention can also be applied to seeds (coating) by either soaking the seeds in a liquid composition or coating them with a solid composition. The composition can also be applied when the propagation material is introduced to the place of cultivation, e.g. when the seeds are sown in the seed furrow. The treatment procedures for plant propagation material and the propagation material thus treated are further objects of the invention.

In the following formulation examples of use in humans, domestic animals, livestock, and pets, the term “active ingredient” is understood to mean one or more enantiomeric active ingredients of formula I or a salt thereof, and preferably the form A of 2-(2,6-difluorophenyl)-4-(4′-trifluoromethylbiphenyl-4-yl)-4,5-dihydro-oxazole.

Tablets: containing one of the active ingredients of formula I can be prepared as follows:

| Composition (for 1000 tablets) | | |
|---|---|---|
| active ingredient of formula I | 25 | g |
| lactose | 100.7 | g |
| wheat starch | 6.25 | g |
| polyethylene glycol 6000 | 5.0 | g |
| talc | 5.0 | g |
| magnesium stearate | 1.8 | g |

| | |
|---|---|
| demineralised water | q.s. |

Preparation: All solid ingredients are first passed through a sieve with a mesh size of 0.6 mm. The active ingredient, the lactose, the talc, and half the starch are then mixed. The other half of the starch is suspended in 40 ml water, and this suspension is added to a boiling solution of the polyethylene glycol in 100 ml water. The resulting starch paste is added to the mixture, and this is then granulated, water being added where appropriate. The granulate is dried overnight at 35°, passed through a sieve with a mesh size of 1.2 mm, mixed with the magnesium stearate, and compressed to form biconcave tablets with a diameter of 6 mm.

Tablets: each containing a total of 0.0183 g active ingredient are prepared as follows:

| Composition (for 10,000 tablets) | |
|---|---|
| active ingredient of formula I | 183.00 g |
| lactose | 290.80 g |
| potato starch | 274.70 g |
| stearic acid | 10.00 g |
| talc | 217.00 g |
| magnesium stearate | 2.50 g |
| colloidal silica | 32.00 g |
| ethanol | q.s. |

A mixture of the active ingredient, the lactose and 274.70 g potato starch is moistened with an ethanolic solution of stearic acid and granulated through a sieve. After drying, the remaining potato starch, the talc, the magnesium stearate, and the colloidal silica are added and the mixture compressed to form tablets of 0.1 g each in weight, which—if so desired—can be scored to allow for a finer adjustment of the dose.

Capsules: each containing a total of 0.022 g active ingredient can be prepared as follows:

| Composition (for 1000 capsules) | |
|---|---|
| active ingredient of formula I | 22.00 g |
| lactose | 249.80 g |
| gelatin | 2.00 g |
| corn starch | 10.00 g |
| talc | 15.00 g |
| water | q.s. |

The active ingredient is mixed with the lactose, the mixture wetted evenly with an aqueous solution of the gelatin and granulated through a sieve with a mesh size of 1.2-1.5 mm. The granulate is mixed with the dried corn starch and the talc, and portions of 300 mg are filled into hard gelatin capsules (size 1).

Premix (feed additive)

| | |
|---|---|
| 0.16 | parts by weight of active ingredient of formula I |
| 4.84 | parts by weight of secondary calcium phosphate, alumina, aerosil, |
| | carbonate or calcium carbonate are mixed until homogeneous with |
| 95 | parts by weight of an animal feed |
| or | |
| 0.41 | parts by weight of active ingredient of formula I |
| 5.00 | parts by weight of aerosil/lime (1:1) are mixed to |
| | homogeneity with |
| 94.59 | parts by weight of a commercial dry food. |

Boli:

| | | |
|---|---|---|
| I | active ingredient | 33.00% |
| | methylcellulose | 0.80% |
| | silicic acid, highly dispersed | 0.80% |
| | corn starch | 8.40% |
| II | lactose, cryst. | 22.50% |
| | corn starch | 17.00% |
| | microcryst. cellulose | 16.50% |
| | magnesium stearate | 1.00% |

The methylcellulose is first stirred into water. After the material has swollen, silicic acid is stirred in and the mixture homogeneously suspended. The active ingredient and the corn starch are mixed. The aqueous suspension is worked into this mixture and kneaded to a dough. The resulting mass is granulated through a 12 M sieve and dried. In a further step, all 4 adjuvants are thoroughly mixed. Finally, the premixtures resulting from the first two partial steps are mixed and compressed to form boli.

| Injectables: | | |
|---|---|---|
| A. | Oily vehicle (slow release) | |
| | active ingredient of formula I | 0.1-1.0 g |
| | groundnut oil | ad 100 ml |
| or | | |
| | active ingredient of formula I | 0.1-1.0 g |
| | sesame oil | ad 100 ml |

Preparation: The active ingredient is dissolved in part of the oil with stirring and where appropriate gentle heating, then cooled and made up to the desired volume and sterile-filtered through a suitable membrane filter with a pore size of 0.22 μm.

### PREPARATION EXAMPLES

### Example P1

Preparation of Enantiomers A and B of 2-(2,6-difluoro-phenyl)-4-(4′-trifluoromethylbiphenyl-4-yl)-4,5-dihydro-oxazole

a) The enantiomer mixture is dissolved in a solvent mixture comprising 40 ml of ethanol and 60 ml of hexane, and chromatographed on a Chiralcel column (OD 10×50 cm) first of all for 120 mins. with a hexane/isopropanol mixture (9:1) at a flow rate of 150 ml/min., then for 80 mins. at a flow rate of 100 ml/min. with pure ethanol. After ca. 31 mins., the maximum peak of enantiomer A of the title compound is attained and after ca. 49 mins., that of enantiomer B is attained.

b) The enantiomer mixture is dissolved in pure ethanol and chromatographed on a Chiralcel column (OJ(1082) 25×0.46 cm) at a flow rate of 1 ml/min. with pure ethanol. After ca. 5.5 mins., the maximum peak of enantiomer A of the title compound is attained and after ca. 7.5 mins., that of enantiomer B is attained.

### Example P2

The other compounds of Table I can also be produced in analogous manner to that of example P1.

TABLE 1

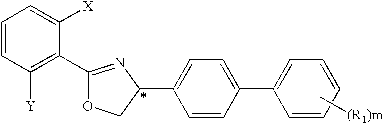

| No. | X | Y | (R₁)ₘ | enantiomer | optical rotation¹ |
|---|---|---|---|---|---|
| 1.1 | F | F | 4-CF₃ | A | −24.3° (20.7 mg) |
| 1.2 | F | F | 4-CF₃ | B | +23.8° (21 mg) |
| 1.3 | F | F | 4-CH₃ | A | |
| 1.4 | F | F | 4-CH₃ | B | |
| 1.5 | F | F | 4-OCF₃ | A | |
| 1.6 | F | F | 4-OCF₃ | B | |
| 1.7 | F | F | 3-CF₃ | A | |
| 1.8 | F | F | 3-CF₃ | B | |
| 1.9 | F | F | 4-OCF₂CHF₂ | A | |
| 1.10 | F | F | 4-OCF₂CHF₂ | B | |
| 1.11 | F | F | 4-OCHF₂ | A | |
| 1.12 | F | F | 4-OCHF₂ | B | |
| 1.13 | F | F | 4-SCF₃ | A | |
| 1.14 | F | F | 4-SCF₃ | B | |
| 1.15 | F | F | 4-CF₂CN | A | |
| 1.16 | F | F | 4-CF₂CN | B | |
| 1.17 | F | F | 3-CH₃, 4-CF₃ | A | |
| 1.18 | F | F | 3-CH₃, 4-CF₃ | B | |
| 1.19 | F | Cl | 4-OCF₃ | A | |
| 1.20 | F | Cl | 4-OCF₃ | B | |

¹α_{D} (589 nm Na_{D}), dissolved in 2 ml methanol

Formulation examples of application in crop protection (%=percentage by weight)

| Example F1: Emulsion concentrates | a) | b) | c) |
|---|---|---|---|
| active ingredient of formula I | 25% | 40% | 50% |
| calcium dodecylbenzenesulphonate | 5% | 8% | 6% |
| castor oil polyethylene glycol ether(36 mols EO) | 5% | — | — |
| tributyl phenol polyethylene glycol ether | — | 12% | 4% |
| (30 mols EO) | | | |
| cyclohexanone | — | 15% | 20% |
| xylene mixture | 65% | 25% | 20% |

Mixing of finely ground active ingredient and adjuvants results in an emulsion concentrate which is diluted with water to yield emulsions of the desired concentration.

| Example F2: Solutions | a) | b) | c) | d) |
|---|---|---|---|---|
| active ingredient of formula I | 80% | 10% | 5% | 95% |
| ethylene glycol monomethyl ether | 20% | — | — | — |
| polyethylene glycol (MW 400) | — | 70% | — | — |
| N-methylpyrrolid-2-one | — | 20% | — | — |
| epoxidised coconut oil | — | — | 1% | 5% |
| petrol (boiling limits: 160-190°) | — | — | 94% | — |

Mixing of finely ground active ingredient and adjuvants results in a solution which is suitable for application in the form of fine droplets.

| Example F3: Granulates | a) | b) | c) | d) |
|---|---|---|---|---|
| active ingredient of formula I | 5% | 10% | 8% | 21% |
| kaolin | 94% | — | 79% | 54% |
| highly dispersed silicic acid | 1% | — | 13% | 7% |
| attapulgite | — | 90% | — | 18% |

The active ingredient is dissolved in dichloromethane, the solution sprayed onto the carrier mixture, and the solvent evaporated off under vacuum.

| Example F4: Dusts | a) | b) |
|---|---|---|
| active ingredient of formula I | 2% | 5% |
| highly dispersed silicic acid | 1% | 5% |
| talc | 97% | — |
| kaolin | — | 90% |

Mixing of active ingredient and carriers results in dusts ready for use.

| Example F5: Wettable powders | a) | b) | c) |
|---|---|---|---|
| active ingredient of formula I | 25% | 50% | 75% |
| sodium ligninsulphonate | 5% | 5% | — |
| sodium lauryl sulphate | 3% | — | 5% |
| sodium diisobutyl naphthalene sulphonate | — | 6% | 10% |
| octylphenol polyethylene glycol ether | — | 2% | — |
| (7-8 mols EO) | | | |
| highly dispersed silicic acid | 5% | 10% | 10% |
| kaolin | 62% | 27% | — |

Active ingredient and adjuvants are mixed and the mixture ground in a suitable mill. Wettable powders are obtained which can be diluted with water to give suspensions of the desired concentration.

| Example F6: Emulsion concentrate | |
|---|---|
| active ingredient of formula I | 10% |
| octylphenol polyethylene glycol ether (4-5 mols EO) | 3% |
| calcium dodecylbenzenesulphonate | 3% |
| castor oil polyethylene glycol ether (36 mols EO) | 4% |
| cyclohexanone | 30% |
| xylene mixture | 50% |

Mixing of finely ground active ingredient and adjuvants results in an emulsion concentrate which is diluted with water to yield emulsions of the desired concentration.

| Example F7: Dusts | a) | b) |
|---|---|---|
| active ingredient of formula I | 5% | 8% |
| talc | 95% | — |
| kaolin | — | 92% |

Ready-to-use dusts are obtained by mixing the active ingredient and carrier, then grinding the mixture in a suitable mill.

| Example F8: Extruder granulate | |
|---|---|
| active ingredient of formula I | 10% |
| sodium lignin sulphonate | 2% |
| carboxymethylcellulose | 1% |
| kaolin | 87% |

Active ingredient and adjuvants are mixed, the mixture ground, moistened with water, extruded and granulated, and the granulate dried in a stream of air.

| Example F9: Coated granulate | |
|---|---|
| active ingredient of formula I | 3% |
| polyethylene glycol (MW 200) | 3% |
| kaolin | 94% |

Homogeneous application of the finely ground active ingredient to the kaolin moistened with polyethylene glycol in a mixer results in dust-free coated granulates.

| Example Fl0: Suspension concentrate | |
|---|---|
| active ingredient of formula I | 40% |
| ethylene glycol | 10% |
| nonylphenol polyethylene glycol ether (15 mols EO) | 6% |
| sodium lignin sulphonate | 10% |
| carboxymethylcellulose | 1% |
| aqueous formaldehyde solution (37%) | 0.2% |
| aqueous silicone oil emulsion (75%) | 0.8% |
| water | 32% |

Mixing of finely ground active ingredient and adjuvants results in a suspension concentrate which is diluted with water to yield suspensions of the desired concentration.

### BIOLOGICAL EXAMPLES

Examples of Use in Crop Protection

### Example B1

Ovicidal Effect on Heliothis virescens

Eggs of Heliothis virescens deposited on filter paper are immersed briefly in a test solution comprising 400 ppm of the active ingredient to be tested in acetone/water. After the test solution has dried, the eggs are incubated in Petri dishes. After 6 days, the percentage hatching rate of the eggs is compared with that for untreated controls (% reduction in hatching rate).

Enantiomers A of table 1 show good efficacy in this test. In particular, enantiomer A of Example P1 shows a response of more than 80%.

### Example B2

Effect on Diabrotica balteata Larvae

Corn seedlings are sprayed with an aqueous emulsion spray mixture containing 400 ppm of active ingredient. After drying of the spray deposit, the corn seedlings are colonised with 10 second instar larvae of Diabrotica balteata and placed in a plastic container. Six days later they are evaluated. The percentage reduction of the population (% response) is determined by comparing the number of dead larvae on the treated plants with those on the untreated plants.

Enantiomers A of table 1 show good efficacy against Diabrotica balteata in this test. In particular, enantiomer A of Example P1 shows a response of more than 80%.

### Example B3

Effect Against Tetranychus urticae

Young bean plants are colonised with a mixed population of Tetranychus urticae and, one day later, are sprayed with an aqueous emulsion spray mixture containing 400 ppm of active ingredient. The plants are subsequently incubated for 6 days at 25° C. and then evaluated. The percentage reduction of the population (% response) is determined by comparing the total number of dead eggs, larvae, and adults on the treated plants with those on the untreated plants.

Enantiomers A of table 1 show good efficacy against Tetranychus urticae in this test. In particular, enantiomer A of Example P1 shows a response of more than 80%.

### Example B4

Effect on Heliothis virescens Caterpillars

Young soya plants are sprayed with an aqueous emulsion spray mixture containing 400 ppm of active ingredient. After drying of the spray deposit, the soya plants are colonised with 10 first-instar larvae of Heliothis virescens and placed in a plastic container. Six days later they are evaluated. The percentage reduction of the population and percentage reduction in feeding damage (% response) is determined by comparing the number of dead larvae and the extent of feeding damage on the treated plants with those on the untreated plants.

Enantiomers A of table 1 show good efficacy against Heliothis virescens in this test. In particular, enantiomer A of Example P1 shows a response of more than 80%.

### Example B5

Effect Against Plutella xylostella Caterpillars

Young cabbage plants are sprayed with an aqueous emulsion spray mixture containing 400 ppm of active ingredient. After the spray coating has dried on, the cabbage plants are colonised with 10 third-instar caterpillars of Plutella xylostella and placed in a plastic container. Three days later they are evaluated. The percentage reduction of the population and percentage reduction in feeding damage (% response) is determined by comparing the number of dead larvae and the extent of feeding damage on the treated plants with those on the untreated plants.

Enantiomers A of table 1 show good efficacy against Plutella xylostella in this test. In particular, enantiomer A of Example P1 shows a response of more than 80%.

### Example B6

Ovicidal/Larvicidal Effect on Heliothis virescens

Eggs of Heliothis virescens laid on cotton are sprayed with an aqueous emulsion spray mixture containing 400 ppm of active ingredient. After 8 days, the percentage hatching rate of the eggs and the survival rate of the caterpillars are compared with those for untreated controls (% reduction of population)

Enantiomers A of table 1 show good efficacy against Heliothis virescens. In particular, enantiomer A of Example P1 shows a response of more than 80%.

### Example B7

Ovicidal Effect on Tetranychus urticae

Young bean plants are colonised with females of Tetranychus urticae, which are removed again after 24 hours. The plants colonised with eggs are sprayed with an aqueous emulsion spray mixture containing 400 ppm of active ingredient. The plants are incubated for 6 days at 25° C. and then evaluated. The percentage reduction of the population (% response) is determined by comparing the total number of dead eggs, larvae, and adults on the treated plants with those on the untreated plants.

Enantiomers A of table 1 show good efficacy against Tetranychus urticae in this test. In particular, enantiomer A of Example P1 shows a response of more than 80%.

### Example B8

Effect Against Panonychus ulmi (Resistant to Organophosphates Und Carbaryl)

Apple seedlings are colonised with adult females of Panonychus ulmi. After seven days, the infected plants are sprayed with an aqueous emulsion spray mixture containing 400 ppm of the test compound until they are dripping wet, and cultivated in the greenhouse. After 14 days, they are evaluated. The percentage reduction of the population (% response) is determined by comparing the number of dead spider mites on the treated plants with those on the untreated plants.

Enantiomers A of table 1 show good efficacy in the above test. In particular, enantiomer A of Example P1 shows a response of more than 80%.

### Example B9

Effect Against Nilaparvata lugens

Rice plants are sprayed with an aqueous emulsion spray mixture containing 400 ppm of active ingredient. After the spray coating has dried on, the rice plants are colonised with second and third instar larvae of plant and leaf-hoppers. 21 days later they are evaluated. The percentage reduction of the population (% response) is determined by comparing the number of surviving plant and leaf-hoppers on the treated plants with those on the untreated plants.

Enantiomers A of table 1 show good efficacy in the above test. In particular, enantiomer A of Example P1 shows a response of more than 80%.

### Example B10

Effect on Spodoptera littoralis

Young soybean plants are sprayed with an aqueous emulsion spray mixture containing 400 ppm of active ingredient. After the spray deposit has dried, the plants are colonised with 10 third-instar larvae of Spodoptera littoralis and placed in a plastic container. Three days later they are evaluated. The percentage reduction of the population and of the feeding damage (% response) is determined by comparing the total number of dead caterpillars and the feeding damage on the treated plants with those on the untreated plants.

Enantiomers A of table 1 show good efficacy in the above test. In particular, enantiomer A of Example P1 shows a response of more than 80%.

### Example B11

Effect Against Aphis craccivora

Pea seedlings are infected with Aphis craccivora, subsequently sprayed with a spray mixture containing 400 ppm of active ingredient, and then incubated at 20° C. 3 and 6 days later, they are evaluated. The percentage reduction of the population (% response) is determined by comparing the number of dead aphids on the treated plants with those on the untreated plants.

Enantiomers A of table 1 show good efficacy in the above test. In particular, enantiomer A of Example P1 shows a response of more than 80%.

### Example B12

Effect Against Crocidolomia binotalis

Young cabbage plants are sprayed with an aqueous emulsion spray mixture containing 400 ppm of active ingredient. After the spray coating has dried on, the cabbage plants are colonised with 10 third-instar caterpillars of Crocidolomia binotalis and placed in a plastic container. Three days later they are evaluated. The percentage reduction of the population and of the feeding damage (% response) is determined by comparing the total number of dead caterpillars and the feeding damage on the treated plants with those on the untreated plants.

Enantiomers A of table 1 show good efficacy in the above test. In particular, enantiomer A of Example P1 shows a response of more than 80%.

### Example B13

Effect Against Anthonomus grandis

Young cotton plants are sprayed with an aqueous emulsion spray mixture containing 400 ppm of active ingredient. After the spray coating has dried on, the cotton plants are colonised with 10 adult Anthonomus grandis and placed in a plastic container. Three days later they are evaluated. The percentage reduction of the population and of the feeding damage (% response) is determined by comparing the total number of dead beetles and the feeding damage on the treated plants with those on the untreated plants.

Enantiomers A of table 1 show good efficacy in the above test. In particular, enantiomer A of Example P1 shows a response of more than 80%.

### Example B14

Effect Against Aonidiella aurantii

Potato tubers are colonised with crawlers of Aonidiella aurantii. After about 2 weeks, the potatoes are immersed in an aqueous emulsion or suspension spray mixture containing 400 ppm of active ingredient. After the tubers have dried off, they are incubated in a plastic container. Evaluation is effected 10 to 12 weeks later by comparing the survival rate of the crawlers of the first secondary generation of the treated population with that of untreated control batches.

Enantiomers A of table 1 show good efficacy in the above test. In particular, enantiomer A of Example P1 shows a response of more than 80%.

### Example B15

Effect Against Bemisia tabaci

Dwarf bean plants are placed in gauze cages and colonised with adults of Bemisia tabaci. Following oviposition, all adults are removed. Ten days later, the plants and the nymphs thereon are sprayed with an aqueous emulsion spray mixture containing 400 ppm of the active ingredient. After a further 14 days, the percentage hatching rate of the eggs is compared with that of untreated controls.

Enantiomers A of table 1 show good efficacy in the above test. In particular, enantiomer A of Example P1 shows a response of more than 80%.

Examples of Use in (Veterinary) Medicine and in the Field of Hygiene

### Example B16

In vitro Effect on Boophilus microplus

Four test series each of 10 engorged female adults of Boophilus microplus are stuck to a plastic plate and covered for 1 hour with a wad of cottonwool soaked with an aqueous suspension or emulsion of the test substance. The test is carried out with concentrations of 100, 32, 10, 3.2, 1.0 and 0.32 ppm. The wad of cottonwool is then removed, and the ticks are incubated for 28 days for the eggs to be laid. The effect on Boophilus microplus is assessed according to the following 5 criteria:

1. Number of dead females (immobile with black discoloration) before ovipositon;

2. Number of ticks surviving for several days, but no eggs laid;

3. Number of cases in which eggs are laid, but nothing is hatched;

4. Number of cases in which eggs are laid, and from which embryos hatch, but which do not develop into larvae;

5. Number of cases in which embryos hatch, develop into larvae, and do not show any anomalies within 4 weeks.

Enantiomers A of formula I in this test show the effect described under point 4.Hatching of larvae is 100% suppressed by these substances at concentrations of 100, 32, 10 and 3.2 ppm. Even at 1 ppm, a 60 to 90% suppression of the hatching rate is observed. Therefore, enantiomer A of 2-(2,6-difluorophenyl)-4-(4′-trifluoromethylbiphenyl-4-yl)-4,5-dihydro-oxazole is the most active test substance. In contrast, enantiomer B of formula I shows practically no activity under the same conditions.

This test is carried out with both the BIARRA and the ULAM strain, and the results in both cases are identical.

### Example B17

Comparative in vitro Effect on Dermanyssus gallinae or the Enantiomers A and B and the Enantiomer of 2-(2,6-difluorophenyl)-4-(4′-trifluoromethylbiphenyl-4-yl)-4,5-dihydrooxazole

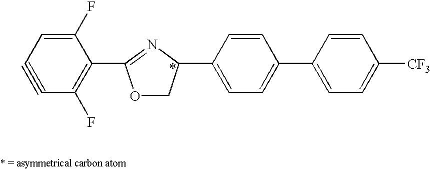

15 fed adult female mites of the genus Dermanyssus gallinae fixed on a plastic adhesive film are brought into contact with 50 μl of an aqueous suspension or emulsion of the test substance. The test is carried out with concentrations of 32, 10, 3.2, 1.0, 0.32 to 0.1 ppm. After drying, the film is stuck onto a glass disc. This creates a kind of air bubble around each mite, the lower surface of which is formed by the glass disc and the upper surface by a bulging of the adhesive film. This bubble contains sufficient air for the mite to avoid suffocating. After 5 days, the effect of the test substance is evaluated with the aid of a stereo-microscope by assessing the effect on mortality, egg deposition, egg quality, hatching rate, pupation rate, and development of protonymphs according to the following 4 criteria:

1. if 9 to 10 mites are dead, this indicates a lethal effect (M);

2. if 2 or more mites survive, but do not produce any eggs, this indicates sterility (S);

3. if 2 or more mites survive and produce eggs, but no larvae hatch from these eggs and no protonymphs develop, this indicates a development-inhibiting effect (H);

4. if 2 or more mites survive and lay the usual number of normal eggs, from which larvae hatch and develop into protonymphs, this indicates no activity.

The racemic mixture shows in this test the effect described under point 1. It completely inhibits the development of protonymphs at concentrations of 0.02 ppm and higher. enantiomer A shows the same effect but already at the very low concentration of 0.0064 ppm and even lower. Enantiomer B shows at concentrations up to 10 ppm absolutely no effect (c.f. point 4) and cannot be distinguished from the untreated control. To reach a considerable efficacy the concentration of enantiomer B has to be at least 20 ppm, and even at this concentration only an activity of type 3 can be reached. To reach an activity of type 1 the concentration of enantiomer B has to be at least 32 ppm. The results are summarized as follows (EC₁₀₀=minimum dosage to reach 100% mortality):

| Test Compound | EC₁₀₀ (ppm) | Type of activity |
|---|---|---|
| Racemate | 0.2 | 1 |
| Enantiomer A | 0.0064 | 1 |
| (Enantiomer B | 20.0 | 3) |
| Enantiomer B | 32.0 | 1 |

This shows that the activity of enantiomer A is more than 30 times higher than the activity of the racemate and even 3000-5000 times higher that the activity of enantiomer B.

### Example B18

In vitro Effect on Australian Sheep Blowfly Lucilia cuprina

In a test tube, 4 ml of a culture medium suitable for blowfly larvae on an agar base is liquefied by heating and mixed with 10 ml of a suspension or emulsion of the test solution. The mixture is left to cool and becomes a solidified culture medium. Test tubes are prepared containing test substances in concentrations of 10, 3.2, 1 and 0.32 ppm. The solidified culture medium is inoculated with 30 to 50 freshly laid eggs of the Lucilia cuprina blowfly, the test tubes are loosely closed with a wad of cottonwool, and cultivated in an incubator at 26 to 28° C. After 4 days, the test tubes are taken from the incubator and the larvicidal effect of the test substances is determined. If large vital larvae in the third stage of development are found in a culture medium which is now liquefied and brownish, this indicates an absence of larvicidal effect. By contrast, if the culture medium is not discoloured and remains solidified, and no larvae are found, this indicates 100% larvicidal activity. Enantiomers A of formula I in this test show a 100% larvicidal effect on blowflies in all test concentrations. In contrast, enantiomer B of formula I shows practically no activity under the same conditions.

### Example B19

Effect Against Blattella germanica

Sufficient acetonic solution (0.1%) of the active ingredient is added to a Petri dish for the quantity thereof to correspond to an application rate of 2 g/m². When the solvent has evaporated, 20 nymphs of Blattella germanica (last nymph stage) are placed in the dish and exposed to the action of the test substance for 2 hours. The nymphs are then anaesthetised with CO₂, added to a fresh Petri dish and kept in the dark at 25° and 50 to 70% humidity. After 48 hours, the insecticidal effect is evaluated by determining the mortality rate.

Enantiomers A of table 1 show good efficacy in the above test. In particular, enantiomer A of Example P1 shows a response of more than 80%.

### Example B20

Effect Against Musca domestica

A sugar cube is treated with a solution of the test substance in such a way that the concentration of test substance in the sugar, after drying over night, is 250 ppm. The cube treated in this way is placed on an aluminium dish with wet cottonwool and 10 adult Musca domestica of an OP-resistant strain. It is covered with a beaker and incubated at 25° C. The mortality rate is determined after 24 hours.

Enantiomers A of table 1 show good efficacy in the above test. In particular, enantiomer A of Example P1 shows a response of more than 80%.

### Example B21

In vitro Effect on Eggs, Larvae, or Pupae of the Cat Flea Ctenocephalides felis

Acetonic test solutions are prepared containing test substances in concentrations of 15, 1.5, 0.15 and 0.015 ppm. 9.9 ml of each test solution is mixed with 14.85 g of culture medium for flea larvae and dried for about 12 hours. The slightly clumped, dry culture medium is mechanically pulverized again until it is homogeneous and free-flowing. It is then transferred to bottles for the breeding of fleas. To each bottle, 100 to 200 flea eggs are added, the bottles are loosely closed with a wad of cottonwool and placed in an incubator at 25 to 26° C. and a relative humidity of about 60%. After 21 days, the effect of the test substances in the different concentrations is evaluated and the lowest effective concentration determined using a stereomicroscope. The activity is evaluated on the basis of the hatching rate, larva development, pupation, and the hatching of young fleas. Enantiomers A of formula (I) show a pronounced effect in this test. Up to a dilution of 10 ppm, the development of young fleas is shown to be completely suppressed. Therefore, enantiomer A of 2-(2,6-difluorophenyl)-4-(4′-trifluoromethylbiphenyl-4-yl)-4,5-dihydro-oxazole is the most active test substance. In contrast, enantiomer B of formula I shows practically no activity under the same conditions.

### Example B22

In vitro Effect on Third-instar Larvae of Haemonchus contortus

2 μl of a 5% solution of the test substance in DSMO or methanol is diluted with a further ml of solvent and test tubes wetted on the inside with the solution. After drying, 2 ml agar agar is added to each test tube. Each test tube is now inoculated with 100 fresh Haemonchus contortus eggs in deionized water, the test tubes are loosely closed with a wad of cottonwool and placed in an incubator at 34 to 36° C. and a relative humidity of about 60 to 100%. 24 hours after hatching of the larvae, 30 μl of a culture medium for bacteria is added so that the bacteria introduced with the eggs can reproduce. The volume of water should be such that the test tubes are about one third full. The effect is assessed on the basis of the hatching rate, the development of third stage larvae, the paralysis or death of larvae, or of other development stages. Enantiomers A of formula I show a pronounced development-inhibiting effect in this test. Up to a dilution of 32 ppm, the development of third stage larvae is shown to be completely suppressed. In contrast, enantiomer B of formula I shows practically no activity under the same conditions.

### Example B23

In vivo Effect of Topical Treatment on Infestation with Mouse Fur Mites

Mice infested with mites (Myocopetes musculinus and Myobia musculi) are anaesthetized, and the density of the mite population is examined under a stereomicroscope. The mice are divided into groups with the same infection index, i.e. with the same mite population in each case, the index consisting of a scale from 1 (no mites) to 30 (greatest mite density). For test purposes, only mice with an index of at least 25 on the said scale (high mite density) are used. The test substance is applied in the form of a pour-on solution, suspension or emulsion, i.e. applied topically to the fur. The dose is in the range 32 to 0.1 mg/kg bodyweight. Per mouse, 150 μl of solution, suspension or emulsion is applied along the topline. Efficacy is evaluated 7, 28 and 56 days after application by comparing the infection index after treatment with that before treatment. The efficacy is expressed as a percentage reduction of the mite population.

Enantiomers A of formula I in this test show a reduction in mite infestation of more than 80% at concentrations up to 10 mg/kg bodyweight. In contrast, enantiomer B of formula I shows practically no activity under the same conditions.

### Example B24

In vivo Effect Against Infestation with Mouse Fur Mites After Subcutaneous Injection

Mice infested with mites (Myocopetes musculinus and Myobia musculi) are anaesthetized, and the density of the mite population is examined under a stereomicroscope. The mice are divided into groups with the same infection index, i.e. with the same mite population in each case, the index consisting of a scale from 1 (no mites) to 30 (greatest mite density). For test purposes, only mice with an index of at least 25 on the said scale (high mite density) are used. The test substance is dissolved in a 2: 3 mixture of glycerol formal and polyethylene glycol and injected subcutaneously into the test animals. The dose is in the range 20 to 0.1 mg/kg bodyweight. Efficacy is evaluated 7, 28 and 56 days after application by comparing the infection index after treatment with that before treatment. The efficacy is expressed as a percentage reduction of the mite population. Enantiomers A of formula I in this test show a reduction in mite infestation of more than 80% at concentrations up to 0.32 mg/kg bodyweight. The mice, however, do not show skin irritations at the injection site or any other unwanted side effects. The substances are shown to be very well tolerated. In contrast, enantiomer B of formula I shows practically no activity under the same conditions.

What is claimed is:

1. An enantiomer of formula 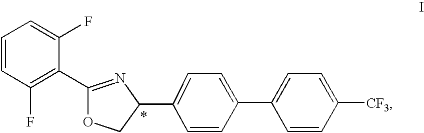
in free form or in salt form, wherein the enantiomer has a negative optical rotation of α_{D} (589 nm Na_{D}) in methanol.

2. A method of producing enantiomers of formula I according claim 1, wherein an enantiomer mixture of formula I is separated using column chromatography on a chiral stationary phase with organic solvents or solvent mixtures and the desired enantiomer isolated or a free enantiomer of formula I obtained according to said method is converted into a salt or a salt of an enantiomer of formula I obtained according to said method is converted into the free compound of formula I or into another salt.

3. A pesticide composition comprising an active agent and an adjuvant, wherein said active agent is consisting essentially of said enantiomer of claim 1, in free form or in agrochemically employable salt form.

4. A method for controlling pests, comprising applying the composition of claim 3 to pests or their habitat.

5. The method according to claim 4 for the control of insects and members of the order Acarina.

6. A method for preparing the composition of claim 3, comprising intimately mixing and/or grinding of said active ingredient with said adjuvant.

7. A method for preparing the pesticide composition of claim 3, which method comprises preparing said enantiomer of formula I according to claim 1, either in free form or in the form of an agrochemically acceptable salt.

8. A method for controlling pests, comprising applying said composition of claim 1.

9. The method of claim 8, wherein said composition is applied on a plant propagation material.

10. The method according to claim 9, comprising treating said propagation material or a locus for cultivation of said propagation material.

11. A plant propagation material, which is treated according to the method according to claim 10.

12. A composition to combat ectoparasites or endoparasites in humans or animals, comprising a pesticidal agent and a physiologically acceptable adjuvant, wherein said pesticidal agent is consisting essentially of said enantiomer of claim 1.

13. A method for controlling ectoparasites or endoparasites in human or animals, comprising the step of applying said enantiomer of claim 1.

14. A pesticide composition comprising an active enantiomer and an adjuvant, wherein said active enantiomer comprises said enantiomer of claim 1 and said enantiomer has a purity of at least 95%.
